SEQUENCE LISTING (57 sequences)

SEQ ID NO: 1 - DNA, 980 bp, Bacillus subtilis
  source 1..980: mol_type = other DNA
ctgacgatattgcctcctgcttttccggccagaccatctttgtaattaatgccggaataagcaactttaatcaggacaccatccttcggcaaatcctctgttgatatggttttcacatggactgaaacatcatcggcatttttttctgcctgcaaggcttgaaataacgttgacattcggcacactccttttcatttatatcgtaaccgaagaacgttcaaaaaaccaaatcatcaagccgccattttcacttcgccggcacattgagacaataatggacaaatccggtatcctcttcatagccgttttgctcatacaagcttcttgccttccggttgtggtgctcagtctgaagtgttaaacattttgccccgttttgccctgcataatcctttgcggcagaaagcagccggccgccggctccctttgtacgcgcatgaggaacgacaaataagtcatttaatatgtatatccttttcattgacacagaagaaaacgttggatagagctgggtaaagcctatgaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcgctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 2 - DNA, 91 bp, synthetic construct
  source 1..91: mol_type = other DNA
gctgataaacagctgacatcaactaaaagtttcattaaatactttgaaaaaagttgttgacttaaaagaagctaaatgttatagtaattgt

SEQ ID NO: 3 - DNA, 58 bp, Bacillus subtilis
  source 1..58: mol_type = other DNA
acagaatagtcttttaagtaagtctactctgaatttttttaaaaggagagggtaaaga

SEQ ID NO: 4 - DNA, 87 bp, Bacillus subtilis
  source 1..87: mol_type = other DNA
gtgagaagcaaaaaattgtggatcagcttgttgtttgcgttaacgttaatctttacgatggcgttcagcaacatgtctgcgcaggct

SEQ ID NO: 5 - DNA, 1122 bp, synthetic construct
  source 1..1122: mol_type = other DNA
aaaaaaactatagttaaacatttaacaaatgtctgataattgtttataatataaatatgcttaatctttattaagcatatcgagtatcgaacggtatatagaaacgcacgattgttttatttgaaaaacgatttaatgtaagcattttctttttggattaaagaagatgtatacatcatgtgttgtttttgctcctgggttttggtttaattttaaaaggggaaatttttaaaaaaagggagagatgagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtagtgactgcagtgagatctggtaatgactctctagcttgaggcatcaaataaaacgaaaggctcagtcgaaagact

SEQ ID NO: 6 - DNA, 96 bp, Bacillus amyloliquefaciens
  source 1..96: mol_type = other DNA
tgaaacgtctcaatcgccgcttcccggtttccggtcagctcaatgccgtaacggtcggcggcgttttcctgataccgggagacggcattcgtaatc

SEQ ID NO: 7 - DNA, 363 bp, synthetic construct
  source 1..363: mol_type = other DNA
tcactgacacacagaaaatttggaggctcaggcggatcaccgttttcaggcctgtcatcaattgcagttagatcaggctcatatctggatgcgattattatcgatggcgttcatcatggcggatcaggcggaaatctgtcaccgacatttacatttggctctggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtat

SEQ ID NO: 8 - protein, 121 aa, synthetic construct
SLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 9 - DNA, 363 bp, synthetic construct
  source 1..363: mol_type = other DNA
tcactgacacacagaaaatttggaggcgatggcggatcaccgttttcaggcctgtcatcaattgcagttagatcaggctcatatctggatgcgattattatcgatggcgttcaacatggcggatcaggcggaaatctgtcaccgacatttacatttaaaccgggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtat

SEQ ID NO: 10 - protein, 121 aa, synthetic construct
SLTHRKFGGDGGSPFSGLSSIAVRSGSYLDAIIIDGVQHGGSGGNLSPTFTFKPGEYISNMTIRSGDYIDNISFETNMGRRFGPYGGSGGSANTLSNVKVIQINGSAGDYLDSLDIYYEQY

SEQ ID NO: 11 - DNA, 363 bp, synthetic construct
  source 1..363: mol_type = other DNA
tcactgacacacagaaaatttggaggcgatggcggatcaccgttttcaggcctgtcatcaattgcagttagatcaggctcatatctggatgcgattattatcgatggcgttcaacatggcggatcaggcggaaatctgtcaccgacatttacatttgaatctggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtat

SEQ ID NO: 12 - protein, 121 aa, synthetic construct
SLTHRKFGGDGGSPFSGLSSIAVRSGSYLDAIIIDGVQHGGSGGNLSPTFTFESGEYISNMTIRSGDYIDNISFETNMGRRFGPYGGSGGSANTLSNVKVIQINGSAGDYLDSLDIYYEQY

SEQ ID NO: 13 - DNA, 363 bp, synthetic construct
  source 1..363: mol_type = other DNA
tcactgacacacagaaaatttggaggcaatggcggatcaccgttttcaggcctgtcatcaattgcagttagatcaggctcatatctggatgcgattattatcgatggcgttcatcatggcggatcaggcggaaatctgtcaccgacatttacatttaaatctggcgaatatatcagcaacatgacaattagaagcggagactatatcgacaacatcagctttgaaacaaattatggcagacgctttggaccgtatggcggaagcggaggcagcgcaaatacactgtcaaatgttaaagtcattcagatcaatggcagcgcaggcgattatctggattcactggatatctattacgaacagtat

SEQ ID NO: 14 - protein, 121 aa, synthetic construct
SLTHRKFGGNGGSPFSGLSSIAVRSGSYLDAIIIDGVHHGGSGGNLSPTFTFKSGEYISNMTIRSGDYIDNISFETNYGRRFGPYGGSGGSANTLSNVKVIQINGSAGDYLDSLDIYYEQY

SEQ ID NO: 15 - DNA, 2077 bp, Bacillus subtilis
  source 1..2077: mol_type = other DNA
tcagccttattctcctgataacgcgagacagcattagaaaaaggcgtaaccgcaaagctcaaaacagaaaacaaaagcaataacagcggaagtgccgcaagatcatgccgcccttctaaatgaaacatgctgcgggttaggcgaaccgtccgcttgtaaagcttatcaatgacataaaatccggcgagcgacacgagcaaatagccagccagaccgatgtaaacgtgcttcatgacataatggcccatttcgtggcccataataaacagaatttctgaatcgtcaagtttgttcagcgtcgtatcccacaatacaatccgtttattggccccaattcctgtaacataggcattcagcgcatttgttttttctgacatgttcacttcatatacatggtcagccggaatattggcttcatctgccagctctaaaattttgctttcaagctctttgtttttcagcggataaaaatcattgtataaaggatcgataatgaccggctgaataaaaaacagaaacagcgaaaacggcactgttaacagccaggcgtataaccaccattttttttcatgccttttgatcagccaataaaaaacgagaacgcaaagcgtaaagattggaaagctgatccaaaagctgataacctgatccttagcccagctggccgttgtctgtgtggaaatgttatagtcaagcgatacttgatagcctatccaatctaaaggcagcgtcaccaatgttgtaatcagcgaaagcacaaacacaaaaccaacggtctgcaaaaaccgaaaaggcacggccgcttcgatccatttcttgattttctttgaaacaccgctgacaagcagaacaaaaaacagaaaccaatcaagtggtaccccgataaaaaataaaaaattcttgacattcgaatactgctcggccactgccaactcagacggcttcatgaaagaagccggatcagcctgcgtccctttcacggcttccggtattgcggaatcacccgacaggaaaaaataccaataaaaaaacagcccgtacagcacgtaagcaagtcctgctgccgcaatccacttgcgcataacgcctcactcctcacatcaacccgttacttctattgtaatcataaattcaaattcttagaaccaagctgtgttccgcacttttccacccttttaagcatggaaaccccgatcgctgggaaaactaacaatgtttggagtgatgcaaatgaaaaaaatagtggcagccatcgtggtaatcggtcttgtgtttatcgcatttttttatctttacagccgatcaggcgatgtgtatcaatcggtagacgcggatttgatcacactgtcttcaagcggccaggaagatatcgagattgaaaaaagacagcacgtcaaagatatgctggatattatgaatcagggaaaacaggtgaagacagaaaaaacatcagcccctgattacgaagggacaatcaagtttcataaagaccggtatgactcattcagactatggattgacggcagccagcaagccgtttttttgaaggatggcacatactacaaattaagcaaaaatgatacaaaggcgctgctaaatattattaaaaaagaagcaaaggattgaaaatgaaaaagcgaagctaaccgcttcgctttttcattttattggggcaaaatatctctcagtgcccgtctgagcattttccccgtcgcatttttcggaatatcgtcaagaaacgtaatggcggcaggccgcttgtattttgccagatgcttttcgcagtgctgcatgatgtcctcctctgttaccccagagcgtttcggcaccacatatccctttaccgcttccccgctttgggggtccggcacgccgatgacaaccgcctccttgacgtccggatggctgtacagcacctcctccacctcccgcggatacacattgtatcctcctacaatgatcatgtcttttttccggtcaacaatgtaaaaatagccgtcctcatcccgtcttgccaagtcccccgtataaagccacccgtcttttaccccacgagagacttaactacgcactagca

SEQ ID NO: 16 - DNA, 1750 bp, Bacillus subtilis
  source 1..1750: mol_type = genomic DNA
tcacgagctgtataccgggtaaagtctccgcagttcagcagctgatcatcctgtccacaaaaaagcttgcagaaaaataacattctctgcaagctgatcctgttaaagcttcacaatcactcttccttgaatgcgattttgcaaaatatcttttaacgcacccggcgtttcttccaatgatacttccctgtccacgatggtcagcagctgatcaggcttgagatcagaagacatgcgctcccaaacagcggctctgacgtccatcggacaatatactgaatcgattccgagcaggcttactccgcgaagaataaaaggatacacggttgccggaacttctcctccgccggttaagccgctcactgcgacagatccgccgtattgaattttgcttaaaagcgaggcaagctgttttccgccgactggatcaaccgctccctgccattgctgcttggacagcgccttaagcgttccgtcatagacatcttccctgctgattacttcgcttgcaccaagctgtttcaaataatcagccgcctcccggtttccggtacttgccaccacatcataaccccgcttgttcagcatcgataccgcaattccgccgacaccgccggttgctcctgtgactagcacgctgcctttttccggagacagaccgttctgttcaagccgatgcactgataacgccgcagtaaatcccgccgttccgtacaccatcgcttcttttaacgaaagattctgtggcaaaggcaccagccagtcaccaggcaccgaagcgtattcacttaatccgccatcacgtgagacaccgagctcatagcttgtcgcgatcacctcatccccctccgcaaaacgcggatcattggaagagacgaccgtacccgcagcatcaatgcctaaaataagcggatactctctgacgatattgcctcctgcttttccggccagaccatctttgtaattaatgccggaataagcaactttaatcaggacaccatccttcggcaaatcctctgttgatatggttttcacatggactgaaacatcatcggcatttttttctgcctgcaaggcttgaaataacgttgacattcggcacactccttttcatttatatcgtaaccgaagaacgttcaaaaaaccaaatcatcaagccgccattttcacttcgccggcacattgagacaataatggacaaatccggtatcctcttcatagccgttttgctcatacaagcttcttgccttccggttgtggtgctcagtctgaagtgttaaacattttgccccgttttgccctgcataatcctttgcggcagaaagcagccggccgccggctccctttgtacgcgcatgaggaacgacaaataagtcatttaatatgtatatccttttcattgacacagaagaaaacgttggatagagctgggtaaagcctatgaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgc

SEQ ID NO: 17 - DNA, 363 bp, synthetic construct
  source 1..363: mol_type = other DNA
tcactgacacacagaaaatttggaggctcaggcggatcaccgttttcaggcctgtcatcaattgcagttagatcaggctcatatctggatgcgattattatcgatggcgttcatcatggcggatcaggcggaaatctgtcaccgacatttacatttggctctggcgaatatatcagcaacatgacaattagaagcggagactatatcgacaacatcagctttgaaacaaataatggcagacgctttggaccgtatggcggaagcggaggcagcgcaaatacactgtcaaatgttaaagtcattcagatcaatggcagcgcaggcgattatctggattcactggatatctattacgaacagtat

SEQ ID NO: 18 - protein, 121 aa, unidentified
SLTHRKFGGSGGSPFSGLSSIAVRSGSYLDAIIIDGVHHGGSGGNLSPTFTFGSGEYISNMTIRSGDYIDNISFETNNGRRFGPYGGSGGSANTLSNVKVIQINGSAGDYLDSLDIYYEQY

SEQ ID NO: 19 - DNA, 738 bp, synthetic construct
  source 1..738: mol_type = other DNA
tcactgacacacagaaaatttggaggctcaggcggatcaccgttttcaggcctgtcatcaattgcagttagatcaggctcatatctggatgcgattattatcgatggcgttcatcatggcggatcaggcggaaatctgtcaccgacatttacatttggctctggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtatggaacaggctcactgacacacagaaaatttggaggctcaggcggatcaccgttttcaggcctgtcatcaattgcagttagatcaggctcatatctggatgcgattattatcgatggcgttcatcatggcggatcaggcggaaatctgtcaccgacatttacatttggctctggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtattaa

SEQ ID NO: 20 - protein, 245 aa, synthetic construct
SLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQYGTGSLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 21 - DNA, 738 bp, synthetic construct
  source 1..738: mol_type = other DNA
tcactgacacacagaaaatttggaggcgatggcggatcaccgttttcaggcctgtcatcaattgcagttagatcaggctcatatctggatgcgattattatcgatggcgttcaacatggcggatcaggcggaaatctgtcaccgacatttacatttaaaccgggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtatggaacaggctcactgacacacagaaaatttggaggcgatggcggatcaccgttttcaggcctgtcatcaattgcagttagatcaggctcatatctggatgcgattattatcgatggcgttcaacatggcggatcaggcggaaatctgtcaccgacatttacatttaaaccgggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtattaa

SEQ ID NO: 22 - protein, 245 aa, synthetic construct
SLTHRKFGGDGGSPFSGLSSIAVRSGSYLDAIIIDGVQHGGSGGNLSPTFTFKPGEYISNMTIRSGDYIDNISFETNMGRRFGPYGGSGGSANTLSNVKVIQINGSAGDYLDSLDIYYEQYGTGSLTHRKFGGDGGSPFSGLSSIAVRSGSYLDAIIIDGVQHGGSGGNLSPTFTFKPGEYISNMTIRSGDYIDNISFETNMGRRFGPYGGSGGSANTLSNVKVIQINGSAGDYLDSLDIYYEQY

SEQ ID NO: 23 - DNA, 735 bp, synthetic construct
  source 1..735: mol_type = other DNA
tcactgacacacagaaaatttggaggcgatggcggatcaccgttttcaggcctgtcatcaattgcagttagatcaggctcatatctggatgcgattattatcgatggcgttcaacatggcggatcaggcggaaatctgtcaccgacatttacatttgaatctggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtatggaacaggctcactgacacacagaaaatttggaggcgatggcggatcaccgttttcaggcctgtcatcaattgcagttagatcaggctcatatctggatgcgattattatcgatggcgttcaacatggcggatcaggcggaaatctgtcaccgacatttacatttgaatctggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtat

SEQ ID NO: 24 - protein, 245 aa, synthetic construct
SLTHRKFGGDGGSPFSGLSSIAVRSGSYLDAIIIDGVQHGGSGGNLSPTFTFESGEYISNMTIRSGDYIDNISFETNMGRRFGPYGGSGGSANTLSNVKVIQINGSAGDYLDSLDIYYEQYGTGSLTHRKFGGDGGSPFSGLSSIAVRSGSYLDAIIIDGVQHGGSGGNLSPTFTFESGEYISNMTIRSGDYIDNISFETNMGRRFGPYGGSGGSANTLSNVKVIQINGSAGDYLDSLDIYYEQY

SEQ ID NO: 25 - DNA, 735 bp, synthetic construct
  source 1..735: mol_type = other DNA
tcactgacacacagaaaatttggaggcaatggcggatcaccgttttcaggcctgtcatcaattgcagttagatcaggctcatatctggatgcgattattatcgatggcgttcatcatggcggatcaggcggaaatctgtcaccgacatttacatttaaatctggcgaatatatcagcaacatgacaattagaagcggagactatatcgacaacatcagctttgaaacaaattatggcagacgctttggaccgtatggcggaagcggaggcagcgcaaatacactgtcaaatgttaaagtcattcagatcaatggcagcgcaggcgattatctggattcactggatatctattacgaacagtatggaacaggctccctgacccatcgtaagttcggtggaaacggaggttccccattctccggactgtcctccatcgctgtgcgttccggatcctacctggatgctatcatcattgatggagtgcatcatggaggttccggaggtaacctgtccccaaccttcaccttcaagtccggagagtacatttctaatatgaccatccgttctggtgattacattgataatatttctttcgagaccaactacggacgtagattcggtccatacggaggttctggtggatctgctaacaccctgtccaacgtgaaggttatccagattaacggatctgctggagattacctggattccctggatatttactatgagcagtac

SEQ ID NO: 26 - protein, 245 aa, synthetic construct
SLTHRKFGGNGGSPFSGLSSIAVRSGSYLDAIIIDGVHHGGSGGNLSPTFTFKSGEYISNMTIRSGDYIDNISFETNYGRRFGPYGGSGGSANTLSNVKVIQINGSAGDYLDSLDIYYEQYGTGSLTHRKFGGNGGSPFSGLSSIAVRSGSYLDAIIIDGVHHGGSGGNLSPTFTFKSGEYISNMTIRSGDYIDNISFETNYGRRFGPYGGSGGSANTLSNVKVIQINGSAGDYLDSLDIYYEQY

SEQ ID NO: 27 - DNA, 1284 bp, Bacillus subtilis
  source 1..1284: mol_type = other DNA
tagacatcgtttccctttagcctttaattttagtatgatatgtaaatgatattgaataaaagctaggaagtgtcgtaatgagcacaaaacctttttacagagatacgtgggcggaaattgacttgtccgcgataaaggaaaatgtcagcaatatgaaaaaacatatcggtgaacatgtccacttgatggcagttgtgaaagcaaacgcctacgggcatggtgatgcagaaacagcaaaggctgctcttgacgcaggtgcttcatgcttggccgtggccattttggatgaagcgatttcactgcgcaaaaagggattgaaggcgcctatattggtgcttggcgcggttcccccggagtatgtggcaatcgctgctgagtatgacgtgaccttaacaggttattctgttgaatggcttcaggaggcagcccgccacacgaaaaaaggttctcttcattttcatctgaaggtcgatacggggatgaacagacttggtgtaaaaacagaggaagaagttcagaacgtgatggcaattcttgaccgcaaccctcgtttaaagtgcaaaggggtatttacccattttgcgacagcggatgaaaaagaaagaggctatttcttaatgcagtttgagcgctttaaagagctgattgctccgctgccgttaaagaatctaatggtccactgcgcgaacagcgccgctggactccggctgaaaaaaggcttttttaatgcagtcagattcggcatcggcatgtatggccttcgcccgtctgctgacatgtcggacgagataccgtttcagctgcgtccggcatttaccctgcattcgacactgtcacatgtcaaactgatcagaaaaggcgagagcgtcagctacggagccgagtacacagcggaaaaagacacatggatcgggacggtgcctgtaggctatgcggacggctggctccgaaaattgaaagggaccgacatccttgtgaagggaaaacgcctgaaaattgccggccgaatttgcatggaccaatttatggtggagctggatcaggaatatccgccgggcacaaaagtcacattaataggccggcagggggatgaatatatttccatggatgagattgcaggaaggctcgaaaccattaactatgaggtggcctgtacaataagttcccgtgttccccgtatgtttttggaaaatgggagtataatggaagtaagaaatcctttattgcaggtaaatataagcaattaacttacctaaaaagcctgagatccctcaggcttttttt

SEQ ID NO: 28 - DNA, 738 bp, synthetic construct
  source 1..738: mol_type = other DNA
tcactgacacacagaaaatttggaggctcaggcggatcaccgttttcaggcctgtcatcaattgcagttagatcaggctcatatctggatgcgattattatcgatggcgttcatcatggcggatcaggcggaaatctgtcaccgacatttacatttggctctggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtatggaggcggttcactgacacacagaaaatttggaggctcaggcggatcaccgttttcaggcctgtcatcaattgcagttagatcaggctcatatctggatgcgattattatcgatggcgttcatcatggcggatcaggcggaaatctgtcaccgacatttacatttggctctggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtattaa

SEQ ID NO: 29 - protein, 245 aa, synthetic construct
SLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQYGGGSLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 30 - DNA, 738 bp, synthetic construct
  source 1..738: mol_type = other DNA
tcactgacacacagaaaatttggaggctcaggcggatcaccgttttcaggcctgtcatcaattgcagttagatcaggctcatatctggatgcgattattatcgatggcgttcatcatggcggatcaggcggaaatctgtcaccgacatttacatttggctctggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtatggacctggctcactgacacacagaaaatttggaggctcaggcggatcaccgttttcaggcctgtcatcaattgcagttagatcaggctcatatctggatgcgattattatcgatggcgttcatcatggcggatcaggcggaaatctgtcaccgacatttacatttggctctggcgaatatatcagcaacatgacaattagaagcggagactatatcgacaacatcagctttgaaacaaatatgggcagacgctttggaccgtatggcggaagcggaggcagcgcaaatacactgtcaaatgttaaagtcattcagatcaatggcagcgcaggcgattatctggattcactggatatctattacgaacagtattaa

SEQ ID NO: 31 - protein, 245 aa, synthetic construct
SLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQYGPGSLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 32 - protein, 6 aa, synthetic construct
YGTTGS

SEQ ID NO: 33 - protein, 16 aa, synthetic construct
SSSGGGGSGGGSSSGS

SEQ ID NO: 34 - protein, 4 aa, synthetic construct
GTTG

SEQ ID NO: 35 - protein, 5 aa, synthetic construct
GTGTG

SEQ ID NO: 36 - protein, 6 aa, synthetic construct
GGSGGS

SEQ ID NO: 37 - protein, 6 aa, synthetic construct
GTGGTG

SEQ ID NO: 38 - protein, 7 aa, synthetic construct
GTGTGTG

SEQ ID NO: 39 - protein, 8 aa, synthetic construct
GTGTTGTG

SEQ ID NO: 40 - protein, 9 aa, synthetic construct
GTGTGTGTG

SEQ ID NO: 41 - protein, 10 aa, synthetic construct
GTGTTGTGTG

SEQ ID NO: 42 - protein, 11 aa, synthetic construct
GTGTGTGTGTG

SEQ ID NO: 43 - protein, 12 aa, synthetic construct
GTGGTGTGTGTG

SEQ ID NO: 44 - protein, 13 aa, synthetic construct
GTGTGTGTGTGTG

SEQ ID NO: 45 - protein, 14 aa, synthetic construct
GTGTGGTGTGTGTG

SEQ ID NO: 46 - protein, 15 aa, synthetic construct
GTGGTGGTGTGTGTG

SEQ ID NO: 47 - protein, 7 aa, synthetic construct
GGSSGGS

SEQ ID NO: 48 - protein, 4 aa, synthetic construct
GSSG

SEQ ID NO: 49 - protein, 5 aa, synthetic construct
GSGSG

SEQ ID NO: 50 - protein, 8 aa, synthetic construct
GGSSGGSG

SEQ ID NO: 51 - protein, 9 aa, synthetic construct
GGSGSGGSG

SEQ ID NO: 52 - protein, 10 aa, synthetic construct
GGSGGSGGSG

SEQ ID NO: 53 - protein, 11 aa, synthetic construct
GGSGGGSGGSG

SEQ ID NO: 54 - protein, 12 aa, synthetic construct
GGSGSGGSGGSG

SEQ ID NO: 55 - protein, 13 aa, synthetic construct
GGSGGSGGSGGSG

SEQ ID NO: 56 - protein, 14 aa, synthetic construct
GGSGSGSGGSGGSG

SEQ ID NO: 57 - protein, 4 aa, synthetic construct
YGTG

(19) United States
(12) Patent Application Publication
(10) Pub. No.: US 2026/0092088 A1
(43) Pub. Date: Apr. 2, 2026

(54) VARIANT ANTI-VIRAL POLYPEPTIDES

(71) Applicant: International N&H Denmark APS, Kongens Lyngby (DK)

(72) Inventors: Cristina BONGIORNI, Fremont, CA (US); Mai DU, Palo Alto, CA (US); David A. ESTELL, San Mateo, CA (US); Keith FROGUE, Arcadia, CA (US); Amy Deming LIU, Sunnyvale, CA (US); Darshana KADEKAR, Bagsvaerd (DE)

(21) Appl. No.: 19/112,643

(22) PCT Filed: Sep. 19, 2023
(86) PCT No.: PCT/US2023/074538

§ 371 Date: Mar. 18, 2025

Related U.S. Application Data
(60) Provisional application No. 63/376,393, filed on Sep. 20, 2022.
(60) Provisional application No. 63/502,297, filed on May 15, 2023.

(51) Int. Cl. C07K 14/405; A61K 38/00; A61P 31/14; C12P 21/00

(52) U.S. Cl. CPC C07K 14/405; A61P 31/14; C12P 21/005; A61K 38/00

(57) ABSTRACT

Provided herein, inter alia, are variant lectin anti-viral polypeptides as well as compositions and methods for expressing and using the same for the treatment and prevention of viral disease in animals.

### CROSS-REFERENCE TO RELATED APPLICATIONS

This application claims priority to U.S. Provisional Patent Application No. 63/376,393, filed Sep. 20, 2022, and to U.S. Provisional Patent Application No. 63/502,297, filed May 15, 2023, the disclosure of each of which is incorporated by reference herein in its entirety.

### FIELD OF THE INVENTION

Provided herein, inter alia, are variant anti-viral polypeptides as well as compositions and methods for expressing and using the same for treatment and prevention of viral disease in animals.

### REFERENCE TO AN ELECTRONIC SEQUENCE LISTING

The contents of the electronic sequence listing (20230831_NB42022-WO-PCT_sequencelisting.xml; Size: 65,998 bytes; and Date of Creation: Aug. 31, 2023) is herein incorporated by reference in its entirety.

### BACKGROUND

Porcine Reproductive and Respiratory Syndrome (PRRS) is one of the most economically important diseases of swine. This disease was first detected in the United States in 1987 (Keffaber 1989) and in Europe in 1990 (Wensvoort et al. 1991). Molecular analysis of the prototype PRRS viruses (PRRSV) VR-2332 and Lelystad (U.S. and European isolates, respectively) has suggested that divergently evolved strains emerged on two continents almost simultaneously, perhaps due to similar changes in swine management practices (Murtaugh et al. 1995; Nelsen et al. 1999).

Since its initial emergence, this virus has spread worldwide, and PRRSV of the European genotype has been detected in U.S. swine herds (Ropp et al. 2004). PRRS is characterized by severe and sometimes fatal respiratory disease and reproductive failure, but also predisposes infected pigs to bacterial pathogens as well as other viral pathogens (Benfield et al. 1992). This factor is a key component of the economically significant Porcine Respiratory Disease Complex (PRDC). The most consistent pathological lesions caused by PRRSV during acute infection are interstitial pneumonia and mild lymphocytic encephalitis. After the acute phase of PRRSV infection, which is typically characterized by viremia and clinical disease, many pigs fully recover yet carry a low-level viral infection for an extended period of time. These “carrier” pigs are persistently infected with PRRSV and shed the virus, either intermittently or continuously, and may infect naïve pigs following direct or indirect contact. Under experimental conditions, persistent infection with PRRSV has been well documented (Albina et al. 1994; Allende et al. 2000; Benfield et al. 1998).

PRRS has and continues to cause significant damage to the global swine industry. What are needed, therefore, are effective and economically sustainable measures to treat or prevent this and other viral diseases in swine and other economically significant livestock.

The subject matter disclosed herein addresses these needs and provides additional benefits as well.

### SUMMARY

Provided herein, inter alia, are compositions and methods for treating or preventing one or more viral diseases in an animal.

Accordingly, in one aspect, provided herein is a non-naturally occurring variant lectin polypeptide or functional fragment thereof comprising an amino acid sequence at least about 60% identical to SEQ ID NO:8 or SEQ ID NO: 18 comprising one or more substitutions at positions 10, 38, 53, 54, and 78. In some embodiments, the polypeptide comprises at least one substitution at position 10. In some embodiments, the substitution at position 10 is selected from the group consisting of X10D, X10E, X10G, X10H, X10N, X10P, X10Q, and X10T. In some embodiments, the substitution at position 10 is selected from the group consisting of S10D, S10E, S10G, S10H, SION, S10P, S10Q, and S10T. In some embodiments of any of the embodiments disclosed herein, the polypeptide comprises at least one substitution at position 38. In some embodiments, the substitution at position 38 is selected from the group consisting of X38P and X38Q. In some embodiments, the substitution at position A31 is selected from the group consisting of H38P and H38Q. In some embodiments of any of the embodiments disclosed herein, the polypeptide comprises at least one substitution at position 53. In some embodiments, the substitution at position 53 is selected from the group consisting of X53A, X53C, X53D, X53E, X53H, X53K, X53L, X53M, X53Q, X53R, X53T, and X53V. In some embodiments, the substitution at position 53 is selected from the group consisting of G53A, G53C, G53D, G53E, G53H, G53K, G53L, G53M, G53Q, G53R, G53T, and G53V. In some embodiments of any of the embodiments disclosed herein, the polypeptide comprises at least one substitution at position 54. In some embodiments, the substitution at position 54 is selected from the group consisting of X54P and X54T. In some embodiments, the substitution at position 54 is selected from the group consisting of S54P and S54T. In some embodiments of any of the embodiments disclosed herein, the polypeptide comprises at least one substitution at position 78. In some embodiments, the substitution at position 78 is selected from the group consisting of X78W, X78H, X78Q, and X78Y. In some embodiments, the substitution at position 78 is selected from the group consisting of M/N78W, M/N78H, M/N78Q, and M/N78Y. In some embodiments of any of the embodiments disclosed herein, the polypeptide comprises at least two substitutions at positions 53 and 78. In some embodiments, the substitution at position 53 is X53E, K, or L; and the substitution at position 78 is X78N or Y. In some embodiments, the substitution at position 53 is G53E or K; and the substitution at position 78 is M/N78Y. In some embodiments of any of the embodiments disclosed herein, the polypeptide comprises at least three substitutions at positions 10, 53, and 78. In some embodiments, the substitution at position 10 is X10D or N; the substitution at position 53 is X53K, E, or L; and the substitution at position 78 is X78Q or Y. In some embodiments, the substitution at position 10 is S10D or N; the substitution at position 53 is G53K, E, or L; and the substitution at position 78 is M/N78Q or Y. In some embodiments of any of the embodiments disclosed herein, the polypeptide comprises at least three substitutions at positions 38, 54, and 78. In some embodiments, the substitution at position 38 is X38Q; the substitution at position 54 is X54P; and the substitution at position 78 is X78Q. In some embodiments, the substitution at position 38 is H38Q; the substitution at position 54 is S54P; and the substitution at position 78 is M/N78Q. In some embodiments of any of the embodiments disclosed herein, the polypeptide comprises at least three substitutions at positions 10, 38, and 53. In some embodiments, the substitution at position 10 is X10N or D; the substitution at position 38 is X38Q; and the substitution at position 53 is X53E, L, or K. In some embodiments, the substitution at position 10 is SION or D; the substitution at position 38 is H38Q; and the substitution at position 53 is G53E, L, or K. In some embodiments of any of the embodiments disclosed herein, the polypeptide comprises at least three substitutions at positions 10, 53, and 54. In some embodiments, the substitution at position 10 is X10N or D; the substitution at position 53 is X53L or K; and the substitution at position 54 is X54P. In some embodiments, the substitution at position 10 is SION; the substitution at position 53 is G53L; and the substitution at position 54 is S54P. In some embodiments of any of the embodiments disclosed herein, the polypeptide comprises at least four substitutions at positions 10, 38, 53, and 54. In some embodiments, the substitution at position 10 is X10N or D; the substitution at position 38 is X38Q; the substitution at position 53 is X53E or K; and the substitution at position 54 is X54P. In some embodiments, the substitution at position 10 is SION or D; the substitution at position 38 is H38Q; the substitution at position 53 is G53E or K; and the substitution at position 54 is S54P. In some embodiments of any of the embodiments disclosed herein, the polypeptide comprises at least four substitutions at positions 10, 53, 54, and 78. In some embodiments, the substitution at position 10 is X10N or D; the substitution at position 53 is X53L or K; the substitution at position 54 is X54P; and the substitution at position 78 is X78Y or Q. In some embodiments, the substitution at position 10 is SION or D; the substitution at position 53 is G53L or K; the substitution at position 54 is S54P; and the substitution at position 78 is M/N78Y or Q. In some embodiments of any of the embodiments disclosed herein, the polypeptide comprises at least four substitutions at positions 10, 38, 53, and 78. In some embodiments, the substitution at position 10 is X10D or N; the substitution at position 38 is X38Q; the substitution at position 53 is X53L, E, or K; and the substitution at position 78 is X78Y or Q. In some embodiments, the substitution at position 10 is S10D or N; the substitution at position 38 is H38Q; the substitution at position 53 is G53L, E, or K; and the substitution at position 78 is M/N78Y or Q. In some embodiments of any of the embodiments disclosed herein, the polypeptide comprises at least five substitutions at positions 10, 38, 53, 54, and 78. In some embodiments, the substitution at position 10 is X10N or D; the substitution at position 38 is X38Q; the substitution at position 53 is X53K or L; the substitution at position 54 is X54P; and the substitution at position 78 is X78Y or Q. In some embodiments, the substitution at position 10 is S10N or D; the substitution at position 38 is H38Q; the substitution at position 53 is G53K or L; the substitution at position 54 is S54P; and the substitution at position 78 is M/N78Y or Q. In some embodiments of any of the embodiments disclosed herein, the polypeptide has antiviral activity. In some embodiments of any of the embodiments disclosed herein, the variant lectin proteins or functional fragment thereof is a fully functional lectin polypeptide that is capable of naturally dimerizing (i.e. forming dimers) with other lectin polypeptides (e.g., variant and/or naturally occurring lectin polypeptides).

In another aspect, provided herein is a polypeptide dimer comprising two polypeptides or functional fragments thereof comprising an amino acid sequence at least about 60% identical to SEQ ID NO:8 or SEQ ID NO: 18. In some embodiments of any of the embodiments disclosed herein, the polypeptide has antiviral activity.

In a further aspect, provided herein is a variant lectin polypeptide dimer comprising two polypeptides or functional fragments thereof comprising any of the variant lectin polypeptides disclosed herein. In some embodiments of any of the embodiments disclosed herein, the two polypeptides in the dimer are identical. In some embodiments of any of the embodiments disclosed herein, the two polypeptides in the dimer are separated by a linker amino acid sequence. In some embodiments, the linker is between 2 and 50 amino acids in length. In some embodiments of any of the embodiments disclosed herein, the linker comprises the amino acid sequence (GTG)ₙ, where n=1-7. In some embodiments of any of the embodiments disclosed herein, the linker comprises (i) any one of SEQ ID NOs:32-57; or (ii) (GGG)ₙ or (GPG)ₙ, where n=1-7. In some embodiments of any of the embodiments disclosed herein, each polypeptide of the polypeptide dimer is capable of dimerization with a Griffithsin protein. In some embodiments of any of the embodiments disclosed herein, the polypeptide has antiviral activity. In some embodiments of any of the embodiments disclosed hereon, the dimer comprises an amino acid sequence selected from the group consisting of SEQ ID NO:20, SEQ ID NO:22, SEQ ID NO:24, SEQ ID NO:26, SEQ ID NO:29, and SEQ ID NO:31.

In yet another aspect, provided herein is a nucleic acid encoding any of the variant lectin polypeptides or variant lectin polypeptide dimers disclosed herein.

In additional aspects, provided herein is a vector comprising any of the nucleic acids disclosed herein.

In still further aspects, provided herein is a recombinant host cell comprising any of the nucleic acids disclosed herein or any of the vectors disclosed herein. In some embodiments, the host cell is a fungal cell, an algal cell, a plant cell, a bacterial cell, or a yeast cell. In some embodiments, the host cell is a Bacillus subtilis cell.

In another aspect, provided herein is a pharmaceutical composition comprising a therapeutically effective amount of any of the variant lectin polypeptides or variant lectin polypeptide dimers disclosed herein and a pharmaceutically acceptable excipient. In some embodiments, the composition is formulated for oral, nasal, and/or topical administration to an animal.

In further aspects, provided herein is a method for treating or preventing a viral infection in an animal, the method comprising administering any of the variant lectin polypeptides or variant lectin polypeptide dimers disclosed herein or any of the pharmaceutical compositions disclosed herein to the animal. In some embodiments, the polypeptide or pharmaceutical composition is administered to the animal via oral, nasal, and/or topical administration. In some embodiments of any of the embodiments disclosed herein, the animal is swine or a horse. In some embodiments, the swine is a sow, gilt, boar, lactation-phase piglet, weaned piglet, and/or finishing pig. In some embodiments of any of the embodiments disclosed herein, the viral infection comprises the virus that causes porcine reproductive and respiratory syndrome (PRRSV), porcine epidemic diarrhea virus (PEDV), porcine rotavirus, or equine viral arteritis (EVA).

In still other aspects, provided herein is a method for producing a polypeptide comprising culturing any of the host cells disclosed herein in a suitable media under conditions suitable for polypeptide expression. In some embodiments, the method further comprises purifying the polypeptide.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 depicts images of SDS-PAGE gels showing the results of a protease stability assay for combinatorial Griffithsin variants.

FIG. 2 is a graph depicting the PRRSV antiviral effect of Griffithsin variants with low antiviral load 1 MOI (Multiplicity of Infection).

FIG. 3 is a graph depicting the PRRSV antiviral effect of Griffithsin variants with high antiviral load 10 MOI (Multiplicity of Infection).

FIG. 4 depicts an image of an SDS-PAGE gel of intracellular samples of Bacillus subtilis nine proteases deleted strains producing Griffithsin M78 monomer and dimers with GGG, GPG, GTG linkers. Lane 1 is See Blue Plus 2 molecular weight ladder; lane 2 is the negative control Bacillus subtilis nine proteases deleted strains; lanes 3, 8, 10 and 12 show the Griffithsin M78 monomer; lanes 4-5 are the Griffithsin M78 GGG dimers; lanes 6-7 are the Griffithsin M78 GPG dimers and lane 9 is the Griffithsin M78 GTG dimer.

FIG. 5 is a graph depicting daily body temperature of pigs from 3 days pre-challenge to 41 days post-challenge. NT/NC=Non treated non challenged pigs; NT/C=Non treated challenged pigs; LDT/C=Challenged pigs receiving low dose of Griffithsin; HDT/C=Challenged pigs receiving high dose of Griffithsin.

FIG. 6 is a bar graph depicting virus load in serum samples at different time points post-challenge. Treatment groups: NT/C=Non treated challenged pigs; LDT/C=Challenged pigs receiving low dose of Griffithsin; HDT/C=Challenged pigs receiving high dose of Griffithsin.

FIG. 7 is a bar graph depicting gross lung scores depicting presence of lesions in different lobes on day 42 post-challenge. Average score from the 7 lobes per pig is shown. Treatment groups: NT/C=Non treated challenged pigs; LDT/C=Challenged pigs receiving low dose of Griffithsin; HDT/C=Challenged pigs receiving high dose of Griffithsin.

FIG. 8 is a bar graph depicting microscopic lung score on day 42 post-challenge. Lung tissues were microscopically evaluated for macrophage infiltration and graded from 0 to 4. 0=no microscopic lesion, 1=mild interstitial pneumonia, 2=moderate multifocal interstitial pneumonia, 3=moderate diffuse interstitial pneumonia and 4=severe interstitial pneumonia. Treatment groups: NT/C=Non treated challenged pigs; LDT/C=Challenged pigs receiving low dose of Griffithsin; HDT/C=Challenged pigs receiving high dose of Griffithsin.

FIG. 9 is a bar graph depicting body weight of pigs recorded at different time points pre- and post-challenge. Treatment groups: NT/NC=Non treated non challenged pigs; NT/C=Non treated challenged pigs; LDT/C=Challenged pigs receiving low dose of Griffithsin; HDT/C=Challenged pigs receiving high dose of Griffithsin.

FIG. 10 is a bar graph depicting average daily body weight gain in pigs. Treatment groups: NT/C=Non treated challenged pigs; LDT/C=Challenged pigs receiving low dose of Griffithsin; HDT/C=Challenged pigs receiving high dose of Griffithsin.

### DETAILED DESCRIPTION

Porcine Reproductive and Respiratory Syndrome (PRRS) is characterized by severe and sometimes fatal respiratory disease and reproductive failure, but also predisposes infected pigs to bacterial pathogens as well as other viral pathogens. Thus, there is a significant need for therapeutics capable of treating and preventing infection and spread of PRRS within the swine livestock industry.

Lectins are generally defined as carbohydrate binding proteins that can recognize and bind simple or complex carbohydrates in a reversible and highly specific manner, while displaying no catalytic activity (Lagarda-Diaz et al., 2017). Lectin proteins were originally named hemagglutinins, due to their ability to agglutinate red blood cells (and other cells). More recently, lectins such as the red algae (Griffithsia sp.) Griffithsin (GRFT) protein have been evaluated for their anti-viral activities (Whitley et al., 2013). However, the economics of recombinant lectin production (e.g., using currently available host expression systems and downstream recovery process thereof) has significantly limited acceptance and/or use of lectins as anti-microbial compositions.

This invention is based, at least in part, on the inventors' discovery that introducing one or more substitutions into the amino acid sequence of a lectin (for example, a Griffithsin (GRFT) lectin polypeptide) can improve one or more properties (such as, without limitation, thermostability, recombinant expression levels, and/or protease stability) of the lectin resulting in improved recombinant production and stability. Further, as shown in the Examples, the engineered lectins possess potent antiviral properties.

### I. Definitions

The term “lectin,” as used herein, refers to a carbohydrate-binding protein.

The term “variant lectin protein” or “variant lectin” as used interchangeably herein refers to a polymer of amino acid residues that has carbohydrate-binding activity and that contains at least one amino acid modification relative to a wild-type (i.e. naturally-occurring) lectin amino acid sequence.

As used herein, “microorganism” or “microbe” refers to a bacterium, a fungus, a virus, a protozoan, and other microbes or microscopic organisms.

The terms “protein” and “polypeptide” refer to compounds comprising amino acids joined via peptide bonds and may be used interchangeably. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Amino acid positions in a given polypeptide sequence can be named by the one letter code for the amino acid, followed by a position number. For example, a glycine (G) at position 87 is represented as “G087” or “G87.”

A “variant polypeptide” (such as a “variant lectin polypeptide” or “variant lectin” as used interchangeably herein, for example a variant Griffithsin polypeptide) or a “non-naturally occurring polypeptide” are used interchangeably herein to refer to polypeptides that have been engineered to possess an amino acid sequence that differs by at least one amino acid from a reference or parental polypeptide sequence. In some embodiments, the reference sequence is the wild-type (i.e. naturally-occurring) amino acid sequence of a lectin. In other embodiments, the reference sequence is SEQ ID NO:8 or SEQ ID NO:18.

As used herein, where “amino acid sequence” is recited it refers to an amino acid sequence of a protein or peptide molecule. An “amino acid sequence” can be deduced from the nucleic acid sequence encoding the protein. However, terms such as “polypeptide” or “protein” are not meant to limit the amino acid sequence to the deduced amino acid sequence but can include posttranslational modifications of the deduced amino acid sequences, such as amino acid deletions, additions, and modifications such as glycosylations and addition of lipid moieties. Also, the use of non-natural amino acids, such as D-amino acids to improve stability or pharmacokinetic behavior falls within the scope of the term “amino acid sequence”, unless indicated otherwise.

The term “mature” form of a protein, polypeptide, or peptide refers to the functional form of the protein, polypeptide, or enzyme without the signal peptide sequence and pro-peptide sequence.

The term “wild-type” in reference to an amino acid sequence or nucleic acid sequence indicates that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant/engineered nucleic acids and protein sequences produced in the laboratory or modification of the wild-type sequence).

The term “sequence identity” or “sequence similarity” as used herein, means that two polynucleotide sequences, a candidate sequence and a reference sequence, are identical (i.e. 100% sequence identity) or similar (i.e. on a nucleotide-by-nucleotide basis) over the length of the candidate sequence. In comparing a candidate sequence to a reference sequence, the candidate sequence may comprise additions or deletions (i.e. gaps) as compared to the reference sequence (which does not comprise additions or deletions) for optimal alignment of the two sequences. Optimal alignment of sequences for determining sequence identity may be conducted using the any number of publicly available local alignment algorithms known in the art such as ALIGN or Megalign (DNASTAR), or by inspection.

The term “percent (%) sequence identity” or “percent (%) sequence similarity,” as used herein with respect to a reference sequence is defined as the percentage of nucleotide residues in a candidate sequence that are identical to the residues in the reference polynucleotide sequence after optimal alignment of the sequences and introducing gaps, if necessary, to achieve the maximum percent sequence identity.

As used herein with regard to amino acid residue positions, “corresponding to” or “corresponds to” or “correspond to” or “corresponds” refers to an amino acid residue at the enumerated position in a protein or peptide, or an amino acid residue that is analogous, homologous, or equivalent to an enumerated residue in a protein or peptide.

As used herein, the terms “recombinant” or “non-natural” refer to an organism, microorganism, cell, nucleic acid molecule, vector and the like that has at least one engineered genetic alteration, or has been modified by the introduction of a heterologous nucleic acid molecule; or refer to a cell (e.g., a host cell) that has been altered such that the expression of a heterologous nucleic acid molecule or an endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules (e.g., polynucleotide constructs) that are not found in identical or homologous form within a native (wild-type) cell, or may provide an altered expression pattern of endogenous genes, such as being over-expressed, under-expressed, minimally expressed, or not expressed at all.

“Recombination”, “recombining” or generating a “recombined” nucleic acid is generally the assembly of two or more nucleic acid fragments wherein the assembly gives rise to a chimeric DNA sequence that would not otherwise be found in the genome.

The term “derived” encompasses the terms “originated”, “obtained,” “obtainable,” and “created,” and generally indicates that one specified material or composition finds its origin in another specified material or composition, or has features that can be described with reference to another specified material or composition.

As used herein, an “endogenous gene” refers to a gene in its natural location in the genome of an organism.

As used herein, a “heterologous” gene, a “non-endogenous” gene, or a “foreign” gene refer to a gene (or gene coding sequence; CDS)/open reading frame; ORF) not normally found in the host organism, but that is introduced into the host organism by gene transfer. The term “foreign” gene(s) comprise native genes (or ORF's) inserted into a non-native organism and/or chimeric genes inserted into a native or nonnative organism.

As used herein, a “heterologous control sequence”, refers to a gene expression control sequence (e.g., promoters, enhancers, terminators, etc.) which does not function in nature to regulate (control) the expression of the gene of interest. Generally, heterologous nucleic acids are not endogenous (native) to the cell, or a part of the genome in which they are present, and have been added to the cell, by infection, transfection, transduction, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase during translocation.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any steps involved in the production of the polypeptide including, but not limited to, transcription, post-transcriptional modification, translation, post-translational modification, secretion and the like.

As used herein, “nucleic acid” refers to a nucleotide or polynucleotide sequence, and fragments or portions thereof, as well as to DNA, cDNA, and RNA of genomic or synthetic origin, which may be double stranded or single-stranded, whether representing the sense or antisense strand. It will be understood that as a result of the degeneracy of the genetic code, a multitude of nucleotide sequences may encode a given protein. It is understood that the polynucleotides (or nucleic acid molecules) described herein include “genes”, “vectors” and “plasmids”. Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

The term “promoter” as used herein refers to a nucleic acid sequence capable of controlling the expression of a coding sequence or functional RNA. In general, a coding sequence is located (3′) downstream to a promoter sequence. Promoters may be derived in their entirety from a native gene or be composed of different elements derived from different promoters found in nature, or even comprise synthetic nucleic acid segments.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation. Thus, a nucleic acid is “operably linked” when it is placed into a functional relationship with another nucleic acid sequence. For example, DNA encoding a secretory leader (e.g., secretory signal sequence) is operably linked to DNA for a polypeptide if it is expressed as a pre-protein that participates in the secretion of the polypeptide; a promoter or enhancer is operably linked to a coding sequence if it affects the transcription of the sequence; a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in a desired host cell. For example, in certain embodiments, the present disclosure is directed to a polynucleotide comprising an upstream (5′) promoter (or 5′ promoter region, or tandem 5′ promoters and the like) functional in a host cell, wherein the promoter region is operably linked to a nucleic acid sequence (e.g., an ORF) encoding a variant lectin or variant lectin dimer protein.

As used herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, transcription leader sequences, RNA processing site, effector binding site and stem-loop structures.

As used herein, the terms “modification” and “genetic modification” are used interchangeably and include, but are not limited to: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption. (c) a gene conversion, (d) a gene deletion, (e) the down-regulation of a gene, (f) specific mutagenesis and/or (g) random mutagenesis of any one or more the genes disclosed herein.

As used herein, “disruption of a gene” or a “gene disruption”, are used interchangeably and refer broadly to any genetic modification that substantially prevents a host cell from producing a functional gene product (e.g., a protein). Thus, as used herein, a gene disruption includes, but is not limited to, frameshift mutations, premature stop codons (i.e., such that a functional protein is not made), substitutions eliminating or reducing activity of the protein (such that a functional protein is not made), internal deletions, insertions disrupting the coding sequence, mutations removing the operable link between a native promoter required for transcription and the open reading frame, and the like.

As used herein, the term “introducing”, as used in phrases such as “introducing into a bacterial cell” or “introducing into a bacterial cell at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection, conjugation and the like.

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). Transformation therefore generally refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein “an incoming sequence” refers to a DNA sequence that is introduced into the host cell chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wildtype gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” or “homology arm” refers to a nucleic acid sequence, which is homologous to a sequence in the host cell's chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, downregulated and the like, according to the invention. These sequences direct where in the host cell's chromosome a DNA construct is integrated and directs what part of the host cell's chromosome is replaced by the incoming sequence. While not meant to limit the present disclosure, a homology box may include about between 1 base pair (bp) to 200 kilobases (kb). In some embodiments, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., ampR, phleoR, specR, kanR, eryR, tetR, cmpR and neoR. Other markers useful in accordance with the invention include, but are not limited to auxotrophic markers, such as serine, lysine, tryptophan; and detection markers, such as β-galactosidase.

As defined herein, a host cell “genome” and the like, includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell. In some embodiments plasmids exist in a parental cell and are lost in the daughter cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophages, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously) or can integrate into a chromosome of a host organism.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. For example, in certain embodiments, a parental Bacillus sp. (host) cell is modified (e.g., transformed) by introducing therein one or more “targeting vectors”.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in some embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked.

A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells. As appreciated by one skilled in the art, many host cells, are generally recognized as safe (GRAS) per US FDA guidelines.

As used herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, heat treatment, ammonium sulphate precipitation or other protein salt precipitation, crystallization, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, a “protein preparation” is any material, typically a solution, generally aqueous, comprising one or more proteins.

As used herein, the terms “broth”, “cultivation broth”, “fermentation broth” and/or “whole fermentation broth” may be used interchangeably and refer to a preparation produced by cellular fermentation that undergoes no processing steps after the fermentation is complete. For example, whole fermentation broths are typically produced when microbial cultures are grown to saturation, incubated under carbon-limiting conditions to allow protein synthesis (e.g., expression of proteins by host cells; and optionally, secretion of the proteins into cell culture medium). Typically, the whole fermentation broth is unfractionated and comprises spent cell culture medium, metabolites, extracellular polypeptides, and microbial cells.

As used herein, the phrase “treated broth” refers to broth that has been conditioned by making changes to the chemical composition and/or physical properties of the broth. Broth “conditioning” may include one or more treatments such as cell lysis, pH modification, heating, cooling, addition of chemicals (e.g., calcium, salt(s), flocculant(s), reducing agent(s), enzyme activator(s), enzyme inhibitor(s), and/or surfactant(s)), mixing, and/or timed hold (e.g., 0.5 to 200 hours) of the broth without further treatment.

As used herein, a “cell lysis” process includes any cell lysis technique known in the art, including but not limited to, enzymatic treatments (e.g., lysozyme, proteinase K treatments), chemical means (e.g., ionic liquids), physical means (e.g., French pressing, ultrasonic), simply holding culture without feeds, and the like.

The terms “recovery,” “recovered,” and “recovering” as used herein refer to at least partial separation of a protein from one or more components of a microbial broth and/or at least partial separation from one or more solvents in the broth (e.g., water or ethanol).

In certain aspects, broths in which host cells have been fermented for the production of variant lectin or variant lectin dimer proteins, with or without broth treatment, are clarified. As used herein, a “clarified” broth means a broth which has been subjected to at least one clarification process to remove cell debris and/or other insoluble components. Clarification processes, as understood in the art include, but are not limited to, centrifugation techniques, cross-flow membrane filtration techniques, solid/liquid filtration techniques, and the like.

“Cell debris” refers to cell walls and other insoluble components that are released or formed after disruption of the cell membrane (e.g., after performing a cell lysis process).

In certain aspects, separation of solvents, as understood in the art include, but are not limited to ultrafiltration, evaporation, spray drying, freezer drying. The obtained solution is referred to as “clarified broth concentrate”, “UF concentrate”, or “ultrafiltrate concentrate”.

As used herein, “performance index” or “PI” refers to calculated activity per unit of an enzyme relative to a parent molecule. In some aspects of any of the embodiments disclosed herein, the parental molecule used in the calculation of the performance index is a non-engineered Griffithsin molecule. In some embodiments, the parental molecule has a performance index of one, by definition. In other embodiments, a performance index greater than one (PI >1.0) indicates improved activity of a variant polypeptide compared to the parent molecule.

As used herein, an “effective amount” or a “therapeutically effective amount” is an amount that provides a nutritional, physiological, or medical benefit to an animal.

The term “animal” as used herein includes all non-ruminant (including humans) and ruminant animals. In a particular embodiment, the animal is a non-ruminant animal, such as a horse and a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment the animal is a ruminant animal including, but not limited to, cattle, young calves, goats, sheep, giraffes, bison, moose, elk, yaks, water buffalo, deer, camels, alpacas, llamas, antelope, pronghom and nilgai.

The term “pathogen” as used herein means any causative agent of disease. Such causative agents can include, but are not limited to, bacterial, viral, fungal causative agents and the like.

A “feed” and a “food,” respectively, means any natural or artificial diet, meal or the like or components of such meals intended or suitable for being eaten, taken in, digested, by a non-human animal and a human being, respectively. As used herein, the term “food” is used in a broad sense—and covers food and food products for humans as well as food for non-human animals (i.e. a feed). The term “feed” is used with reference to products that are fed to animals in the rearing of livestock. The terms “feed” and “animal feed” are used interchangeably. In a preferred embodiment, the food or feed is for consumption by non-ruminants and ruminants.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Compositions

### A. Variant Lectin Polypeptides

Lectins are proteins (or glycoproteins) that possess non-catalytic carbohydrate-binding sites. As generally understood in the art, lectins differ from enzymes because their carbohydrate-binding properties never change, and they are unlike antibodies because they are not induced as an immune response. For example, some of the most well-known lectins are found in leguminous seeds, which are believed to be responsible for innate immunity and defense mechanisms in plants (Peumans and Van Damme, 1998). More recently, the use of lectins in mitigating viral infections (e.g., HIV, MERSCoV, SARS-CoV-2, HCV, Ebola and the like) has received significant attention (PCT Publication No. WO2005/118627, WO2007/064844, WO2010/01424, WO2016/130628, WO2019/108656 and US Publication No. US20110263485). However, the economics of recombinant lectin production (e.g., using currently available host expression systems and downstream recovery process thereof) has significantly limited acceptance and/or use of lectins as anti-microbial compositions.

PCT Publications WO2005/118627 and WO2007/064844 describe methods for isolating the native Griffithsin (GRFT) lectin from the red algae (Griffithsia sp.), cloning the wild-type (grft) gene thereof, generating recombinant polynucleotides thereof, fermenting and producing the same in E. coli host cells, followed by isolating the recombinant His-tagged GRFT protein from the E. coli host, and characterizing its anti-viral activity. However, as described in WO2005/118627 and WO2007/064844, the recombinant GRFT protein (and a C-terminal His-tagged GRFT protein thereof) encoded by the nucleic acids of Example 2, did not efficiently translocate to the periplasmic fraction of E. coli following GRFT protein expression, wherein the majority of the produced GRFT proteins accumulated in the inclusion bodies of E. coli, without the cleavage of the pelB signal sequence located at the N-terminus of the Griffithsin protein. Thus, steps were taken to express Griffithsin in the cytosolic fraction of E. coli, using the N-terminal (His) tagged GRFT, or His-tagged variants of GRFT.

Likewise, PCT Publication No. WO2010/01424 generally describes methods of inhibiting a hepatitis C viral infection of a host comprising administering to the host an effective amount of a glycosylation resistant GRFT protein (or a polypeptide conjugate thereof) in combination with another antiviral protein. For example, as described in this publication, the inventors noted that the anti-viral protein combination of scytovirin (SVN) and Griffithsin (GRFT) have (nanomolar) activity against the Hepatitis C virus (HCV). US Patent Publication No. US20110263485 further describes methods of inhibiting a human immunodeficiency virus (HIV) viral infection of a host comprising administering to the host an effective amount of a gp120 Griffithsin and a peptide selected from a gp41-binding protein, a CCR5-binding protein, a gp120-binding protein, or another Griffithsin, which combinations are potent inhibitors to HIV infection.

PCT Publication No. WO2016/130628 discloses variant Griffithsin proteins having mutations that change the isoelectric point of the GRFT protein, which are reported to alter its solubility in various pH ranges allowing for improved product release.

PCT Publication No. WO2019/108656 generally describes microbicidal compositions comprising an endosperm extract and an anti-HIV lectin, an anti-HIV antibody, or antigen binding antibody fragment thereof. More particularly, the inventors utilized transgenic plants expressing two or more cyanovirin-N (CVN) proteins, Griffithsin (GRFT) proteins, scytovirin (SVN) proteins, other anti-HIV lectin proteins. However, as stated in WO2019/108656, the production of such microbicidal components is expensive because fermenter based expression platforms are required, and the downstream processing facilities must be compliant with good manufacturing practice (GMP) to ensure the removal of viruses or endotoxins, wherein the capacity, scalability and cost issues affecting fermenters are exacerbated when two or three separate products with individual manufacturing processes are required for each microbicide.

The recombinant production of GRFT in tobacco plants (Nicotiana benthamiana) has been described by O'Keefe et al. (2009), wherein the GRFT accumulates to a level of about 1 gram of recombinant GRFT per kilogram of Nicotiana benthamiana leaf material, when expressed via an infectious tobacco mosaic virus (TMV) based vector. For example, as contemplated in the O'Keefe et al. publication, despite the promise that biologics such as Griffithsin have as HIV prophylactics, their practical application as topical microbicides is hampered by high production costs, wherein it is unlikely that any manufacturing system reliant on growth in sterile conditions can be competitive with the price of a male condom, which is necessary if the product is to be available for use by those at risk for sexual transmission of HIV.

Thus, as contemplated and described herein, certain embodiments of the disclosure are related to, inter alia, nucleic acids encoding variant lectin proteins (for example, Griffithsin protein), recombinant cells expressing/producing one or more variant lectin proteins, the recovery of variant lectin proteins, the purification of variant lectin proteins, lectin (protein) preparations and the like. More particularly, in certain embodiments, variant lectin proteins and/or DNA (nucleic acid) sequences encoding the same, may be derived/obtained from known lectin proteins. In certain aspects, variant lectin proteins are derived from a host organism which naturally produces the lectin protein. Thus, in certain embodiments, a variant lectin protein of the disclosure is derived from a eukaryotic cell or a cyanobacterial cell. In certain aspects, a eukaryotic cell is photosynthetic plant cell or an insect cell.

In certain embodiments, a variant lectin protein is derived from one or more of the antiviral lectins described in US Patent Publication Nos. US20040204365, US20020127675, US20110189105 and US20110263485, and/or PCT Publication Nos. WO2005/118627, WO2008/022303, WO2010/014248, WO2014/197650, WO2016/130628 and WO2019/108656 (each incorporated herein by reference in its entirety). Thus, in certain aspects, a variant lectin protein is derived from a scytovirin (SVN), a Griffithsin (GRFT), a cyanovirin-N(CVN), and/or functional fragments thereof.

In other embodiments, a variant lectin protein is derived from one or more of the antiviral lectins described in PCT Publication No. WO2019/108656, such as the Artocarpus heterophyllus (jacalin) lectin, the Musa acuminata (banana) lectin, the Boodlea coacta lectin, the Microcystis viridis lectin, etc.) and/or functional fragments thereof that retain the ability to bind to carbohydrates on viral envelopes described therein.

In other embodiments, a variant lectin protein is derived from a eukaryotic lectin source described in Singh and Sarathi (2012), including but not limited to, the Aaptos papillera (Sponge) lectin, the Abrus precatorius (Jequirty bean) lectin, the Aegapodium podagraria (Ground elder) lectin, the Agaricus bisporus (Common mushroom) lectin, the Albizzia julibrissin (Mimosa tree seed) lectin, the Allomyrina dichotoma (Japanese beetle) lectin, the Aloe arborescens (Aloe plant) lectin, the Amphicarpaea bracteata (Hog peanut) lectin, the Anguilla (Eel) lectin, the Aplysia depilans (Mollusca; Mediterranean sea) lectin, the Arachis hypogaea (Peanut) lectin, the Artocarpus heterophullus (Jacalin) lectin, the Bauhinia purpurea (Camel's foot tree) lectin, the Bryonia diocia (White bryony) lectin, the Caragana arborescens (Siberian pea tree) lectin, the Carcinoscorpius rotundacauda (Horseshoe crab) lectin, and/or functional fragments thereof.

In certain aspects, a variant lectin protein of the disclosure may be classified into groups, including, but not limited to, “galactose (Gal)” specific lectins, “glucose (Glu)” specific lectins, “fucose (Fuc)” specific lectins, “mannose (Man)” specific lectins, “N-acetylgalactosamine (GalNAc)” specific lectins, “Nacetylglucosamine (GluNAc)” specific lectins, “sialic acid” specific lectins, and the like.

Thus, in certain aspects, variant lectins suitable for use according of the instant disclosure may be derived/isolated from eukaryotic lectin source organisms. For example, in certain embodiments, a lectin (protein) can be isolated from the eukaryotic (source) organism using affinity chromatography processes known to one of skill in the art (i.e., one of the aforementioned carbohydrate moieties (Gal, Man, GalNAc, etc.) are attached the inert (chromatographic) matrix such that lectin proteins having binding specificity to the carbohydrate moiety will be retained.

In certain other embodiments, a variant lectin protein is a microvirin (MVN) lectin derived from the cyanobacterium Microcystis aeruginosa (PCC7806), which MVN lectin comprises mannose-specific affinity. In other embodiments, a variant lectin protein is a scytovirin (SVN) derived from the cyanobacterium Scytonema varium, which binds with high affinity to mannose residues on the envelope glycoproteins of viruses and inhibits the virus replication (e.g., see Breitenbach Barroso Coelho et al., 2018). In yet other aspects, a lectin protein is an ESA-2 lectin derived from the red alga Eucheuma serra (e.g., see Breitenbach Barroso Coelho et al., 2018). In another embodiment, a lectin protein is a BanLec (jacalin-related) lectin derived from the fruit of bananas (Musa acuminate), which recognizes high-mannose glycans found on viral envelopes (e.g., see Breitenbach Barroso Coelho et al., 2018).

In certain other embodiments, a variant lectin protein is a mannose-binding plant lectin derived from the rhizomes of Aspidistra elatior (AEL) which has been demonstrated to have significant in vitro inhibitory activity against the vesicular stomatitis virus, Coxsackie virus B4 and respiratory syncytial virus (e.g., see Breitenbach Barroso Coelho et al., 2018).

In certain other embodiments, a lectin protein is a CVL lectin (β-galactose-specific) derived from the marine worm Chaetopterus variopedatus (e.g., see Breitenbach Barroso Coelho et al., 2018).

In certain other embodiments, a variant lectin protein of the disclosure may be derived from the seeds of Vicia faba (fava bean). Lens culinaris (lentil), and Pisum sativum (pea), as generally described in El-Araby et al., 2020 (incorporated herein by reference). As generally set forth in the El-Araby et al. (2020) reference, crude extracts of the three leguminous were purified by affinity chromatography using mannose agarose, wherein the purified fava bean, lentil, and pea lectins comprised molecular weights of 18 kDa, 14 kDa, and 17 kDa, respectively, as determined by amino acid sequence analysis. For example, the minimum inhibitory concentration (MIC) values of these purified lectins when tested against bacteria (Pseudomonas aeruginosa, Staphylococcus aureus, Klebsiella pneumonia) and fungi (Candida albicans) ranged from 1.95 μg/ml to 250 μg/ml.

As contemplated herein, one or more variant lectins may be assessed for function or activity by means including, but not limited to, hemagglutination activity assays, carbohydrate/glycan binding affinity assays, antimicrobial inhibition assays, combinations thereof and the like, as set forth and described in El-Araby et al. (2020). Thus, in certain aspects, variant lectins of the disclosure comprise antimicrobial activity (e.g., antiviral activity, antifungal activity, antibacterial activity).

In other embodiments, provided herein are isolated variant lectin proteins or functional fragment thereof comprising an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO: 18, wherein the variant lectin comprises one or more (such as 1, 2, 3, 4, or 5) substitutions at positions 10, 38, 53, 54, and 78. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise one or more substitutions at positions 10, 38, 53, 54, and 78. In one embodiment the variant lectin exhibits an improvement in thermostability (such as at least about a 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%. 125%, 130%, 135%, 140%, 145%, or 150%, or more, including any values falling in between these percentages, improvement in thermostability) in comparison to a parent lectin protein which does not comprise one or more substitutions at positions 10, 38, 53, 54, and 78. In another embodiment the variant lectin exhibits an improvement in expression (for example, recombinant expression; such as at least about a 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, or 150% or more, including any values falling in between these percentages, improvement in expression) in comparison to a parent lectin protein which does not comprise one or more substitutions at positions 10, 38, 53, 54, and 78. In yet another embodiment, the variant lectin exhibits an improvement in protease stability (such as at least about a 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, or 150% or more, including any values falling in between these percentages, improvement in protease stability) in comparison to a parent lectin protein which does not comprise one or more substitutions at positions 10, 38, 53, 54, and 78. In some embodiments, the variant lectin protein or functional fragment thereof comprises the amino acid sequence of one of SEQ ID NOs: 10, 12, or 14. Thermostability, expression, and protease stability can be assessed via any means known in the art, including those shown herein in Examples 2 and 3.

In other embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%. 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18, wherein the variant lectin comprises a substitution at position 10. The substitution can be one of X10D, X10E, X10G, X10H, X10N, X10P, X10Q, or X10T. In some embodiments, the substitution is selected from the group consisting of S10D, S10E, S10G, S10H, SION, S10P, S10Q, and S10T. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise a substitution at position 10.

In further embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18, wherein the variant lectin comprises a substitution at position 38. The substitution can be one of X38P or X38Q. In some embodiments, the substitution is selected from the group consisting of H38P and H38Q. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO:18) which does not comprise a substitution at position 38.

In still other embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18, wherein the variant lectin comprises a substitution at position 53. The substitution can be one of X53A, X53C, X53D, X53E, X53H, X53K, X53L, X53M, X53Q, X53R, X53T, or X53V. In some embodiments, the substitution is selected from the group consisting of G53A, G53C, G53D, G53E, G53H, G53K, G53L, G53M, G53Q, G53R, G53T, and G53V. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise a substitution at position 53.

In further embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18, wherein the variant lectin comprises a substitution at position 54. The substitution can be one of X54P or X54T. In some embodiments, the substitution is selected from the group consisting of S54P and S54T. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO:18) which does not comprise a substitution at position 54.

In another embodiment, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18, wherein the variant lectin comprises a substitution at position 78. The substitution can be one of X78W, X78H, X78Q, or X78Y. In some embodiments, the substitution is selected from the group consisting of M/N78W, M/N78H, M/N78Q, and M/N78Y. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise a substitution at position 78.

In still further embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%. 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18 and comprises at least two substitutions at positions 53 and 78. The substitution at position 53 can be X53E, K, or L; and the substitution at position 78 can be X78N or Y. In other embodiments, the substitution at position 53 is G53E or K; and the substitution at position 78 is M/N78Y. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise substitutions at positions 53 and 78.

In other embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18 and comprises at least three substitutions at positions 10, 53 and 78. The substitution at position 10 can be X10D or N; the substitution at position 53 can be X53K, E, or L; and the substitution at position 78 can be X78Q or Y. In other embodiments, the substitution at position 10 is S10D or N; the substitution at position 53 is G53K, E, or L; and the substitution at position 78 is M/N78Q or Y. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise substitutions at positions 10, 53 and 78.

In other embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18 and comprises at least three substitutions at positions 38, 54, and 78. The substitution at position 38 can be X38Q; the substitution at position 54 can be X54P; and the substitution at position 78 can be X78Q. In other embodiments, the substitution at position 38 is H38Q; the substitution at position 54 is S54P; and the substitution at position 78 is M/N78Q. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise substitutions at positions 38, 54, and 78.

In other embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18 and comprises at least three substitutions at positions 10, 38, and 53. The substitution at position 10 can be X10N or D; the substitution at position 38 can be X38Q; and the substitution at position 53 can be X53E, L, or K. In other embodiments, the substitution at position 10 is S10N or D; the substitution at position 38 is H38Q; and the substitution at position 53 is G53E, L, or K. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise substitutions at positions 10, 38, and 53.

In other embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:8 and comprises at least three substitutions at positions 10, 53, and 54. The substitution at position 10 can be X10N or D; the substitution at position 53 can be X53L or K; and the substitution at position 54 can be X54P. In other embodiments, the substitution at position 10 is S10N; the substitution at position 53 is G53L; and the substitution at position 54 is S54P. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise substitutions at positions 10, 53, and 54.

In other embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18 and comprises at least four substitutions at positions 10, 38, 53, and 54. The substitution at position 10 can be X10N or D; the substitution at position 38 can be X38Q; the substitution at position 53 can be X53E or K; and the substitution at position 54 can be X54P. In other embodiments, the substitution at position 10 is S10N or D; the substitution at position 38 is H38Q; the substitution at position 53 is G53E or K; and the substitution at position 54 is S54P. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise substitutions at positions 10, 38, 53, and 54.

In other embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18 and comprises at least four substitutions at positions 10, 53, 54, and 78. The substitution at position 10 can be X10N or D; the substitution at position 53 can be X53L or K; the substitution at position 54 can be X54P; and the substitution at position 78 can be X78Y or Q. In other embodiments, the substitution at position 10 is S10N or D; the substitution at position 53 is G53L or K; the substitution at position 54 is S54P; and the substitution at position 78 is M/N78Y or Q. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise substitutions at positions 10, 53, 54, and 78.

In other embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18 and comprises at least four substitutions at positions 10, 38, 53, and 78. The substitution at position 10 can be X10D or N; the substitution at position 38 can be X38Q; the substitution at position 53 can be X53L, E, or K; and the substitution at position 78 can be X78Y or Q. In other embodiments, the substitution at position 10 is S10D or N; the substitution at position 38 is H38Q; the substitution at position 53 is G53L, E, or K; and the substitution at position 78 is M/N78Y or Q. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO:18) which does not comprise substitutions at positions 10, 38, 53, and 78.

In other embodiments, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18 and comprises at least five substitutions at positions 10, 38, 53, 54, and 78. The substitution at position 10 can be X10N or D; the substitution at position 38 can be X38Q; the substitution at position 53 can be X53K or L; the substitution at position 54 can be X54P; and the substitution at position 78 can be X78Y or Q. In other embodiments, the substitution at position 10 is SION or D; the substitution at position 38 is H38Q; the substitution at position 53 is G53K or L; the substitution at position 54 is S54P; and the substitution at position 78 is M/N78Y or Q. In some embodiments, the variant lectin polypeptide exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise substitutions at positions 10, 38, 53, 54, and 78.

In some embodiments, any of the variant lectin proteins or functional fragment thereof disclosed herein is a fully functional lectin polypeptide that is capable of naturally dimerizing (i.e. forming dimers) with other lectin polypeptides. In additional embodiments, the variant lectin proteins or functional fragment thereof disclosed herein have not been specifically engineered into a “monomeric lectin” form which prevents the natural formation of dimers (see, e.g., International Patent Application Publication No. WO2014197650, incorporated by reference herein, which describes lectin polypeptides that have been engineered to be incapable of forming a dimer with another lectin polypeptide by, for example, inserting up to four amino acids at the dimerization site (such as the dimerization site located at Ser16 and Gly17 in SEQ ID NOs: 8 and 18; see also Moulaei et al., 2010, Structure, 18:1104-15 and Moulaei et al., 2015, Retrovirology, 12:6, incorporated by reference herein).

### B. Variant Lectin Polypeptide Dimers

Any of the variant lectin polypeptides disclosed herein can also be dimerized to form lectin polypeptide dimers. As used herein, the phrase “polypeptide dimer” refers to two proteins (or functional fragments thereof) bound together by one or more peptide bonds. In some embodiments, the variant lectin polypeptides of the polypeptide dimer are identical while in other embodiments, each variant can have unique substitutions relative to the other variant in the dimer.

Optionally, the variant lectin polypeptides of the polypeptide dimer can be separated by a linker. “Linker” or “peptide linker,” as used interchangeably herein, generally refer to a synthetic amino acid sequence that connects or links two polypeptide sequences, e.g., that link two variant lectin polypeptides. A linker may connect two amino acid sequences via peptide bonds. In some embodiments, a linker of the present disclosure connects a biologically active moiety to a second moiety in a linear sequence. In some embodiments, the linker can be from 1 to 50 or more amino acids in length (such as any of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more amino acids in length). In some embodiments, the linker comprises one of SEQ ID NOs: 32-57. In other embodiments, the linker comprises one of (GGG)ₙ or (GPG)ₙ, where n=1-8 (such as any of 1, 2, 3, 4, 5, 6, 7, or 8).

In some embodiments, provided herein are isolated variant lectin polypeptide dimers or functional fragment thereof comprising two amino acid sequences at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 and/or SEQ ID NO:18, wherein each variant lectin component of the dimer comprises one or more (such as 1, 2, 3, 4, or 5) substitutions at positions 10, 38, 53, 54, and 78. It should further be understood that each variant lectin component of the dimer can possess unique substitutions relative to the other member. In some embodiments, the variant lectin polypeptide dimer exhibits an improvement in one or more properties including, but not limited to, improved thermostability, improved expression, and/or improved protease stability in comparison to a parent lectin protein (e.g. SEQ ID NO:8 or SEQ ID NO: 18) which does not comprise one or more substitutions at positions 10, 38, 53, 54, and 78 and/or is not a polypeptide dimer. In one embodiment the variant lectin polypeptide dimer exhibits an improvement in thermostability (such as at least about a 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%. 140%, 145%, or 150%, or more, including any values falling in between these percentages, improvement in thermostability) in comparison to a parent lectin protein which does not comprise one or more substitutions at positions 10, 38, 53, 54, and 78 and/or is not a polypeptide dimer. In another embodiment the variant lectin exhibits an improvement in expression (for example, recombinant expression; such as at least about a 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, or 150% or more, including any values falling in between these percentages, improvement in expression) in comparison to a parent lectin protein which does not comprise one or more substitutions at positions 10, 38, 53, 54, and 78 and/or is not a polypeptide dimer. In yet another embodiment, the variant lectin exhibits an improvement in protease stability (such as at least about a 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, or 150% or more, including any values falling in between these percentages, improvement in protease stability) in comparison to a parent lectin protein which does not comprise one or more substitutions at positions 10, 38, 53, 54, and 78 and/or is not a polypeptide dimer. In some embodiments, the variant lectin protein or functional fragment thereof comprises the amino acid sequence of one of SEQ ID NOs: 20, 22, 24, 26, 29, or 31. Thermostability, expression, and protease stability can be assessed via any means known in the art, including those shown herein in Examples 2 and 3.

In some embodiments, each variant lectin polypeptide of the polypeptide dimer disclosed herein is a fully functional lectin polypeptide that is capable of naturally dimerizing (i.e. forming dimers) with other lectin polypeptides (i.e. in the case of a lectin polypeptide dimer, each member of the dimer can further dimerize with another lectin polypeptide). In additional embodiments, each variant lectin polypeptide of the polypeptide dimer disclosed herein have not been specifically engineered into a “monomeric lectin” form which prevents the natural formation of dimers (see, e.g., International Patent Application Publication No. WO2014197650, incorporated by reference herein, which describes lectin polypeptides that have been engineered to be incapable of forming a dimer with another lectin polypeptide by, for example, inserting up to four amino acids at the dimerization site (such as the dimerization site located at Ser16 and Gly17 in SEQ ID NOs: 8 and 18; see also Moulaei et al., 2010, Structure, 18:1104-15 and Moulaei et al., 2015, Retrovirology, 12:6, incorporated by reference herein).

### C. Nucleic Acids and Vectors

In another aspect provided herein is any isolated, recombinant, substantially pure, synthetically derived, or non-naturally occurring nucleic acid comprising a nucleotide sequence encoding any variant lectin polypeptide or a variant lectin polypeptide dimer disclosed herein, and which possesses, at a minimum, anti-viral activity.

Also, of interest is a vector comprising a polynucleotide encoding a variant lectin polypeptide or a variant lectin polypeptide dimer disclosed herein. It will be apparent to the skilled person that the vector can be any suitable expression vector and that the choice of vector may vary depending upon the type of cell into which the vector is to be inserted. Suitable vectors include pGAPT-PG, pRAX1, pGAMD, pGPT-pyrG1, pC194, pJH101, pE194, and pHP13 (See, Harwood and Cutting [eds.], Chapter 3, Molecular Biological Methods for Bacillus, John Wiley & Sons [1990]). See also, Perego, Integrational Vectors for Genetic Manipulations in Bacillus subtilis, in Sonenshein et al., [eds.]Bacillus subtilis and Other Gram-Positive Bacteria: Biochemistry, Physiology and Molecular Genetics. American Society for Microbiology, Washington, D.C. [1993], pp. 615-624), and p2JM103BBI.

The expression vector can be one of any number of vectors or cassettes useful for the transformation of suitable production hosts known in the art. Typically, the vector or cassette will include sequences directing transcription and translation of the relevant gene, a selectable marker, and sequences allowing autonomous replication or chromosomal integration. Suitable vectors generally include a region 5′ of the gene which harbors transcriptional initiation controls and a region 3′ of the DNA fragment which controls transcriptional termination. Both control regions can be derived from homologous genes to genes of a transformed production host cell and/or genes native to the production host, although such control regions need not be so derived.

DNA fragments which control transcriptional termination may also be derived from various genes native to a preferred production host cell. In certain embodiments, the inclusion of a termination control region is optional. In certain embodiments, the expression vector includes a termination control region derived from the preferred host cell.

The expression vector can be included in the production host, particularly in the cells of microbial production hosts. The production host cells can be microbial hosts found within the fungal or bacterial families and which grow over a wide range of temperature, pH values, and solvent tolerances. For example, it is contemplated that any of bacteria, algae, and fungi such as filamentous fungi and yeast may suitably host the expression vector.

Inclusion of the expression vector in the production host cell may be used to express the protein of interest so that it may reside intracellularly, extracellularly, or a combination of both inside and outside the cell. Extracellular expression renders recovery of the desired protein from a fermentation product more facile than methods for recovery of protein produced by intracellular expression.

The recombinant expression vector may be any vector such as a plasmid or virus which can conveniently be subjected to recombinant DNA procedures and lead to expression of the nucleotide sequence. The vector choice will typically depend on the compatibility of the vector with the production host into which the vector is to be introduced. The vectors may be linear or closed circular plasmids. The vector may be an autonomously replicating vector, i.e., a vector, which exists as an extrachromosomal entity, the replication of which is independent of chromosomal replication, e.g., a plasmid, an extrachromosomal element, a minichromosome, or an artificial chromosome. The vector may contain any means for assuring self-replication.

Alternatively, the vector may be one which, when introduced into the production host, is integrated into the genome and replicated together with the chromosome(s) into which it has been integrated. Some non-limiting examples of such vectors is provided in the Fungal Genetics Stock Center Catalogue of Strains (FGSC, <www.fgsc.net), Additional examples of suitable expression and/or integration vectors are provided in Sambrook et al., (1989) supra, Ausubel (1987) supra, van den Hondel et al. (1991) in Bennett and Lasure (Eds.) MORE GENE MANIPULATIONS IN FUNGI, Academic Press. 396-428 and U.S. Pat. No. 5,874,276.

Particularly useful vectors include pTREX, pFB6, pBR322, PUCI8, pUCIO0 and pENTR/D. Suitable plasmids for use in bacterial cells include pBR322 and pUC19 permitting replication in E. coli and pE194 for example permitting replication in Bacillus. Briefly with respect to production in production host cells reference can be made to Sambrook et al., (1989) supra, Ausubel (1987) supra, van den Hondel et al. (1991) in Bennett and Lasure (Eds.) MORE GENE MANIPULATIONS IN FUNGI, Academic Press (1991) pp. 76 and 396-428; Nunberg et al., (1984) Mol. Cell Biol. 4:2306-2315; Boel et al., (1984) 30 EMBO 1 3:1581-1585; Finkelstein in BIOTECHNOLOGY OF FILAMENTOUS FUNGI, Finkelstein et al. Eds. Butterworth-Heinemann, Boston, MA (1992), Chap. 6; Kinghorn et al. (1992) APPLIED MOLECULAR GENETICS OF FILAMENTOUS FUNGI, Blackie Academic and Professional, Chapman and Hall, London; Kelley et al., (1985) EMBO 1 4:475-479; Penttila et al., (1987) Gene 61: 155-164; and U.S. Pat. No. 5,874,276.

A list of suitable vectors may be found in the Fungal Genetics Stock Center Catalogue of Strains (FGSC, www at fgsc.net) and in the Bacillus Genetic Stock Center (BGSC, Hypertext Transfer Protocol Secure://bgsc.org). Suitable vectors include those obtained from for example Invitrogen Life Technologies and Promega. Specific vectors suitable for use in fungal host cells include vectors such as pFB6, pBR322, pUC 18, pUC100, pDONTm201, pDONRTm221, pENTRTm, pGEM(D3Z and pGEM(D4Z.

The vector system may be a single vector or plasmid or two or more vectors or plasmids which together contain the total DNA to be introduced into the genome of the host cell, or a transposon.

The vector may also contain one or more selectable markers to permit easy selection of the transformed cells. A selectable marker is a gene, the product of which provides for biocide or viral resistance and the like. Examples of selectable markers include ones which confer antimicrobial resistance. Nutritional markers also find use in the present invention including those markers known in the art as amdS, argB and pyr4. In some embodiments, the expression vectors will also include a replicon, a gene encoding antibiotic resistance to permit selection of bacteria that harbor recombinant plasmids, and unique restriction sites in nonessential regions of the plasmid to allow insertion of heterologous sequences. The particular antibiotic resistance gene chosen is not critical; any of the many resistance genes known in the art are suitable.

The vector may also contain an element(s) permitting stable integration of the vector into the product host genome or autonomous replication of the vector in the production host independent of the genome of the cell. For integration into the host cell genome, the vector may rely on the nucleotide sequence encoding the aspartic protease or any other element of the vector for stable integration of the vector into the genome by homologous or nonhomologous recombination.

For autonomous replication, the vector may further comprise an origin of replication enabling the vector to replicate autonomously in the production host.

More than one copy of the nucleotide sequence encoding a variant lectin polypeptide or a variant lectin polypeptide dimer disclosed herein may be inserted into the production host to increase production of the variant lectin polypeptide or variant lectin polypeptide dimer. An increase in the copy number of the nucleotide sequence can be obtained by integrating at least one additional copy of the sequence into the genome of the production host or by including an amplifiable selectable marker gene, and thereby additional copies of the nucleotide sequence can be selected for by culturing the production host cells in the presence of an appropriate selectable agent.

A vector comprising the nucleotide sequence encoding a variant lectin polypeptide or a variant lectin polypeptide dimer disclosed herein is introduced into the production host so that the vector is maintained as a chromosomal integrant or as a self-replicating extra-chromosomal vector. Integration is generally considered to be an advantage as the nucleotide sequence is more likely to be stably maintained in the production host. Integration of the vector into the production host chromosome may occur by homologous or non-homologous recombination as was discussed above.

Exemplary vectors include, but are not limited to, pGXT (the same as the pTTTpyr2 vector as described in published PCT application WO2015/017256). There can also be mentioned standard bacterial expression vectors including bacteriophages X and M13, as well as plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Examples of suitable expression and/or integration vectors are provided in Sambrook et al., (1989) supra, Bennett and Lasure (Eds.) More Gene Manipulations in Fungi, (1991) Academic Press pp. 70-76 and pp. 396-428 and articles cited therein; U.S. Pat. No. 5,874,276 and Fungal Genetic Stock Center Catalogue of Strains, (FGSC).

Useful vectors may be obtained from Promega and Invitrogen. Some specific useful vectors include pBR322, pUC18, pUC100, pDONTm201, pENTRTm, pGEN(11)3Z and pGEN4D4Z. However, other forms of expression vectors which serve equivalent functions and which are, or become, known in the art can also be used. Thus, a wide variety of host/expression vector combinations may be employed in expressing the DNA sequences disclosed herein. Useful expression vectors, for example, may consist of segments of chromosomal, non-chromosomal and synthetic DNA sequences such as various known derivatives of 5V40 and known bacterial plasmids, e.g., plasmids from E. coli including col El, pCR1, pBR322, pMb9, pUC 19 and their derivatives, wider host range plasmids, e.g., RP4, phage DNAs e.g., the numerous derivatives of phage λ, e.g., NM989, and other DNA phages, e.g., M13 and filamentous single stranded DNA phages, yeast plasmids such as the 2.mu plasmid or derivatives thereof.

### D. Host Cells

An isolated cell, either comprising a DNA construct or an expression vector, is advantageously used as a host cell in the recombinant production of a variant lectin polypeptide or a variant lectin polypeptide dimer disclosed herein. The cell may be transformed with the DNA construct encoding a variant lectin polypeptide or a variant lectin polypeptide dimer disclosed herein, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector in connection with the different types of host cells.

In certain embodiments, the host cell expresses a heterologous polynucleotide encoding a variant lectin polypeptide or variant lectin polypeptide dimer disclosed herein, or a functional variant thereof. In certain aspects, heterologous polynucleotides encoding variant lectin polypeptides are expression cassettes introduced into the recombinant cell. In certain embodiments, at least one expression cassette is introduced in the host cell. In other embodiments, at least two expression cassettes are introduced in the host cell. Thus, in certain aspects host cells of the disclosure comprise one or more variant lectin polypeptide or variant lectin polypeptide dimer expression cassette introduced therein, wherein the host cells express the variant lectin polypeptide or variant lectin polypeptide dimer when cultivated under suitable conditions.

In certain aspects, the host cell is a Gram-positive bacterial cell and includes the classes Bacilli, Clostridia and Mollicutes (e.g., including Lactobacillales with the families Aerococcaceae, Carnobacteriaceae, Enterococcaceae, Lactobacillaceae, Leuconostocaceae, Oscillospiraceae, Streptococcaceae and the Bacillales with the families Alicyclobacellaceae, Bacillaceae, Caryophanaceae, Listeriaceae, Paenibacillaceae, Planococcaceae, Sporolactobacillaccae, Staphylococcaccae, Thermoactinomycetaceae, Turicibacteraceae).

In certain embodiments, species of the family Bacillaceae include Alkalibacillus, Amphibacillus, Anoxybacillus, Bacillus, Caldalkalibacillus, Cerasilbacillus, Exiguobacterium, Filobacillus, Geobacillus, Gracilibacillus, Halobacillus, Halolactibacillus, Jeotgalibacillus, Lentibacillus, Marinibacillus, Oceanobacillus, Ornithinibacillus, Paraliobacillus, Paucisalibacillus, Pontibacillus, Pontibacillus, Saccharococcus, Salibacillus, Salinibacillus, Tenuibacillus, Thalassobacillus, Ureibacillus, and Virgibacillus.

In other embodiments, a Bacillus sp. cell includes, but is not limited to, B. acidiceler, B. acidicola, B. acidocaldarius, B. acidoterrestris, B. aeolius, B. aerius, B. aerophilus, B. agaradhaerens. B. agri, B. aidingensis, B. akibai, B. alcalophilus, B. algicola, B. alginolyticus, B. alkalidiazo-trophicus, B. alkalinitrilicus, B. alkalitelluris, B. altitudinis, B. alveayuensis, B. alvei, B. amylolyticus, B. aneurinilyticus, B. aneurinolyticus, B. anthracia, B. aquimaris, B. arenosi, B. arseniciselenatis, B. arsenicoselenatis, B. arsenicus, B. arvi, B. asahii, B. atrophaeus, B. aurantiacus, B. axarquiensis, B. azotofixans, B. azotoformans, B. badius, B. barbaricus, B. bataviensis, B. beijingensis, B. benzoevorans, B. bogoriensis, B. boroniphilus, B. borstelenis, B. butanolivorans, B. carboniphilus, B. cecembensis, B. cellulosilyticus, B. centrosporus, B. chagannorensis, B. chitinolyticus, B. chondroitinus, B. choshinensis, B. cibi, B. circulans,

B. clarkii, B. clausii, B. coagulans, B. coahuilensis, B. cohnii, B. curdianolyticus, B. cycloheptanicus, B. decisifrondis, B. decolorationis, B. dipsosauri, B. drentensis, B. edaphicus, B. ehimensis, B. endophyticus, B. farraginis, B. fastidiosus, B. firnmus, B. plexus, B. foraminis, B. fordii, B. formosus, B. fortis, B. fumarioli, B. funiculus, B. fusiformis, B. galactophilus, B. galactosidilyticus, B. gelatini, B. gibsonii, B. ginsengi, B. ginsengihumi, B. globisporus, B. globisporus subsp. globisporus, B. globisporus subsp. inarinus, B. glucanolyticus, B. gordonae, B. halmapalus, B. haloalkaliphilus, B. halodenitrificans, B. halodurans, B. halophilus, B. hemicellulosilyticus, B. herbersteinensis, B. horikoshii, B. horti, B. hemi, B. hwajinpoensis, B. idriensis, B. indicus, B. infantis, B. infernus, B. insolitus, B. isabeliae, B. jeotgali, B. kaustophilus, B. kobensis, B. koreensis, B. kribbensis, B krulwichiae, B. laevolacticus, B. larvae, B. laterosporus, B. lautus, B. lehensis, B. lentimorbus, B. lentus, B. litoralis, B. luciferensis, B. macauensis, B. macerans, B. macquariensis, B. macyae, B. malacitensis, B. mannanilyticus, B. marinus, B. inarisflavi, B. marisinortui, B. massiliensis, B. methanolicus, B. migulanus, B. mojavensis, B. mucilaginosus, B. muralis, B. murimartini, B. mycoides, B. naganoensis, B. nealsonii, B. neidei. B, niabensis, B. niacini, B. novalis, B. odysseyi, B. okhensis, B. okuhidensis, B. oleronius, B. oshimensis, B. pabuli, B. pallidus, B. pallidus (illeg.), B. panaciterrae, B. pantothenticus, B. parabrevis, B. pasteurii, B. patagoniensis, B. peoriae, B. plakortidis, B. pocheonensis, B. polygoni, B. polymyxa, B. popilliae, B. pseudalcaliphilus, B. pseudofirmus, B. pseudomycoides, B. psychrodurans, B. psychrophilus, B. psychrosaccarolyticus, B. psychrotolerans, B. pulvifaciens, B. pycnus, B. qingdaonensis, B. reuszeri, B. runs, B. safensis, B. salarius, B. salexigens, B. saliphilus, B. schlegelii, B. selenatarsenatis, B. selenitrireducens, B. seohaeanensis, B. shackletonii, B. silvestris, B. simplex, B. siralis, B. smithii, B. soli, B. sonorensis, B. sphaericus, B. sporothermodurans, B. stearothermophilus, B. stratosphericus, B. subterraneus, B. subtilis subsp. spizizenii, B. subtilis subsp. subtilis, B. taeanensis, B. tequilensis, B. thermantarcticus, B. thermoaerophilus, B. thermoanmylovorans, B. thermoantarcticus, B. thermocatenulatus, B. thermocloacae, B. thermodenitrificans, B. thermoglucosidasius, B. thernoleovorans, B. thernmoruber, B. thermosphaericus, B. thiaminolyticus, B. thioparans, B. thuringiensis, B. tusciae, B. validus, B. vallismortis, B. vedderi, B. velezensis, B. vietnamensis, B. vireti, B. vulcani, B. wakoensis and B. weihenstephanensis.

In a particular embodiment, the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. As used herein, the “Bacillus genus” include Bacillus sp. that have been reclassified, including, but not limited to B. stearothermophilus, which is now named “Geobacillus stearothermophilus”.

Other examples of suitable bacterial host organisms include Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram-negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species.

Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A glucoamylase expressed by a fungal host cell can be glycosylated, i.e., will comprise a glycosyl moiety. The glycosylation pattern can be the same or different as present in the wild-type glucoamylase. The type and/or degree of glycosylation may impart changes in enzymatic and/or biochemical properties.

As generally set forth above and further described below in the Examples, certain embodiments of the disclosure are related to recombinant host cells expressing heterologous variant lectin polypeptides or variant lectin polypeptide dimer proteins, recombinant polynucleotides (e.g. vectors, expression cassettes) encoding heterologous variant lectin polypeptides or variant lectin polypeptide dimer proteins particularly suitable for introducing (e.g., transforming) into host cells (i.e., for the expression of heterologous variant lectin polypeptides or variant lectin polypeptide dimer proteins) and the like.

In other embodiments, host cells of the disclosure are rendered deficient in the production of one or more native (endogenous) proteins. In certain aspects, recombinant host cells of the disclosure comprise deletions or disruptions of one or more endogenous genes encoding one or more proteases native to the recombinant cell. For example, in certain embodiments, recombinant host cells rendered deficient in the production of one or more native (endogenous) proteases may be used to mitigate variant lectin polypeptide or variant lectin polypeptide dimer proteins degradation (e.g., during fermentation and/or downstream processing of the variant lectin polypeptide or variant lectin polypeptide dimer proteins). It is also contemplated herein that recombinant host cells rendered deficient in the production of one or more native background proteases, or other problematic (native) background proteins, will facilitate variant lectin polypeptide or variant lectin polypeptide dimer protein downstream recovery and purification (e.g., by reducing undesired host cell background (native) protein contaminants).

Thus, certain embodiments are related to, inter alia, nucleic acids, polynucleotides (e.g., plasmids, vectors, expression cassettes), regulatory elements, and the like, suitable for use in constructing recombinant host cells. Accordingly, as presented in the Examples and generally described herein, recombinant cells of the disclosure may be constructed by one of skill using standard and routine recombinant DNA and molecular cloning techniques well known in the art. Methods for genetically modifying cells include, but are not limited to, (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion. (d) a gene deletion, (e) a gene down-regulation, (f) site specific mutagenesis and/or (g) random mutagenesis.

In certain embodiments, recombinant (modified) cells of the disclosure may be constructed by reducing or eliminating the expression of a gene, using methods well known in the art, for example, insertions, disruptions, replacements, or deletions. The portion of the gene to be modified or inactivated may be, for example, the coding region or a regulatory element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other embodiments a modified cell is constructed by gene deletion to eliminate or reduce the expression of the gene. Gene deletion techniques enable the partial or complete removal of the gene(s), thereby eliminating their expression, or expressing a non-functional (or reduced activity) protein product.

In such methods, the deletion of the gene(s) may be accomplished by homologous recombination using a plasmid that has been constructed to contiguously contain the 5′ and 3′ regions flanking the gene. By way of example, the contiguous 5′ and 3′ regions may be introduced into a cell (e.g., on a temperature sensitive plasmid such as pE194) in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is affected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers.

Thus, a person of skill in the art may readily identify nucleotide regions in the gene's coding sequence and/or the gene's non-coding sequence suitable for complete or partial deletion.

In other embodiments, a modified cell is constructed by introducing, substituting, or removing one or more nucleotides in the gene or a regulatory element required for the transcription or translation thereof.

For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art. Thus, in certain embodiments, a gene of the disclosure is inactivated by complete or partial deletion.

In another embodiment, a modified cell is constructed by the process of gene conversion. For example, in the gene conversion method, a nucleic acid sequence corresponding to the gene(s) is mutagenized in vitro to produce a defective nucleic acid sequence, which is then transformed into the host cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is affected by selection for the marker under conditions not permitting plasmid replication. Selection for a second recombination event leading to gene replacement is affected by examination of colonies for loss of the selectable marker and acquisition of the mutated gene. Alternatively, the defective nucleic acid sequence may contain an insertion, substitution, or deletion of one or more nucleotides of the gene, as described below.

In other embodiments, a modified cell is constructed by established anti-sense techniques using a nucleotide sequence complementary to the nucleic acid sequence of the gene. More specifically, expression of the gene by a host cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to, RNA interference (RNAi), small interfering RNA (siRNA), microRNA (miRNA), antisense oligonucleotides, and the like, all of which are well known to the skilled artisan.

In yet other embodiments, a modified cell is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods. Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions and selecting for mutant cells exhibiting reduced or no expression of the gene.

PCT Publication No. WO2003/083125 (incorporated by reference herein) discloses methods for modifying Bacillus cells, such as the creation of Bacillus deletion strains and DNA constructs using PCR fusion to bypass E. coli. PCT Publication No. WO2002/14490 (incorporated by reference herein) discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion.

Those of skill in the art are well aware of suitable methods for introducing polynucleotide sequences into bacterial cells (e.g., E. coli, Bacillus sp.). Indeed, such methods as transformation including protoplast transformation and congression, transduction, and protoplast fusion are known and suited for use in the present disclosure. Methods of transformation are particularly suitable to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified host strain by methods known in the art. In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in host cells are generally known on one of skill in the art. Promoter sequences of the disclosure are generally chosen so that they are functional in the host cells. For example, promoters useful for driving gene expression in Bacillus cells include, but are not limited to, the B. subtilis alkaline protease (aprE) promoter, the α-amylase promoter (amyE) of B. subtilis, the α-amylase promoter (amyL) of B. licheniformis, the α-amylase promoter of B. amyloliquefaciens, the neutral protease (nprE) promoter from B. subtilis, a mutant aprE promoter, or any other promoter from B licheniformis or other related Bacilli. Methods for screening and creating promoter libraries with a range of activities (promoter strength) in Bacillus cells is described in PCT Publication No. WO2002/14490 (incorporated by reference herein).

### E. Anti-Viral Compositions

Any of the anti-viral variant lectin polypeptides or variant lectin polypeptide dimers disclosed herein for use in the methods disclosed herein can be formulated in compositions (for example, pharmaceutical or nutritional compositions). In some embodiments, a herein described variant lectin polypeptide or variant lectin polypeptide dimer having anti-viral activity is useful in treating and/or preventing diseases associated with viral infection (such as, without limitation, porcine reproductive and respiratory syndrome (PRRSV), porcine epidemic diarrhea virus (PEDV), porcine rotavirus, or equine viral arteritis (EVA)). In one embodiment, the invention provides a composition, preferably a pharmaceutical composition. comprising a protein according to the invention. Said pharmaceutical composition optionally comprises a pharmaceutical acceptable carrier, diluent or excipient.

The composition can be presented in any form, for example as a tablet, as an injectable fluid or as an infusion fluid etc. Moreover, the composition, protein, nucleotide and/or vector according to the invention can be administered via different routes, for example topically, intravenously, rectally, bronchially, nasally, or orally. Yet another suitable route of administration is the use of a duodenal drip.

In a one embodiment, the used route of administration is the intravenous route. It is clear for the skilled person, that preferably an effective amount of a variant lectin polypeptide or variant lectin polypeptide dimer according to the invention is delivered. Another suitable route, is the subcutaneous route. If the intravenous route of administration is used, a protein according to the invention can be (at least for a certain amount of time) applied via continuous infusion.

Said composition according to the invention can optionally comprise pharmaceutically acceptable excipients, salts, stabilizers, activators, carriers, permeators, propellants, disinfectants, diluents and preservatives. Suitable excipients are commonly known in the art of pharmaceutical formulation and may be readily found and applied by the skilled artisan, references for instance Remmington's Pharmaceutical Sciences, Mace Publishing Company, Philadelphia Pa., 17th ed. 1985.

For oral administration, the variant lectin polypeptide or variant lectin polypeptide dimer can, for example, be administered in solid dosage forms, such as capsules, tablets (e.g., with an enteric coating), and powders, or in liquid dosage forms, such as elixirs, syrups. and suspensions. Variant lectin polypeptides or variant lectin polypeptide dimers can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable colour, taste, stability, buffering capacity, dispersion or other known desirable features are red iron oxide, silica gel, sodium lauryl sulphate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase palatability.

Enteric coatings arrest the release of the active compound from orally ingestible dosage forms. Depending upon the composition and/or thickness, the enteric coatings are resistant to stomach acid for required periods of time before they begin to disintegrate and permit slow release of the variant lectin polypeptide or variant lectin polypeptide dimer in the lower stomach, small intestines, or large intestine. Examples of some enteric coatings are disclosed in U.S. Pat. No. 5,225,202 (incorporated by reference). Examples of enteric coatings comprise beeswax and glyceryl monostearate; beeswax, shellac and cellulose, optionally with neutral copolymer of polymethacrylicacid esters; copolymers of methacrylic acid and methacrylic acid methylesters or neutral copolymer of polymethacrylic acid esters containing metallic stearates (for references enteric coatings see: U.S. Pat. Nos. 4,728,512, 4,794,001, 3,835,221, 2,809,918, 5,225,202, 5,026,560, 4,524,060, 5,536,507). Most enteric coating polymers begin to become soluble at pH 5.5 and above, with a maximum solubility rates at pH above 6.5. Enteric coatings may also comprise subcoating and outer coating steps, for instance for pharmaceutical compositions intended for specific delivery in the lower GI tract, i.e. in the colon (pH 6.4 to 7.0, ileum pH 6.6), as opposed to a pH in the upper intestines, in the duodenum of the small intestines the pH ranges 7.7-8 (after pancreatic juices and bile addition). The pH differences in the intestines may be exploited to target the enteric-coated variant lectin or variant lectin dimer composition to a specific area in the gut. It also allows the selection of a specific variant lectin or variant lectin dimer that is most active at a particular pH in the intestine.

Any of the variant lectin polypeptides or variant lectin polypeptide dimers disclosed herein can also be administered intranasally. As used herein, “nasal administration” means the same as “intranasal administration” wherein the composition is administered to the interior of the nasal cavity. In some embodiments, the intranasal dosage composition further comprises a mucoadhesive agent. The mucoadhesive agent imparts a mucoadhesive property to the composition so that the composition remains in the nasal cavity and does not drip out of the nose either from the external nares (nostrils) or the back of the throat. Nasal Mucociliary Clearance is one of the limiting factors for nasal drug delivery, because it reduces the time allowed for drug absorption. Thus, improving nasal drug absorption can be achieved by prolonging the contact time between the drug and the nasal mucosa. Mucoadhesion implies the attachment of the composition to the nasal mucus membranes, involving an interaction between mucin and a mucoadhesive agent, a synthetic or natural polymer. The sequential events that occur during mucoadhesion include a first step where the mucoadhesive agent absorbs water from the nasal mucosa and swells. The mucoadhesive agent then intimately penetrates into the nasal mucosa and, hence, localizes the formulation in nasal cavity, enhancing the drug concentration gradient across the epithelium.

Exemplary mucoadhesive agents include an alginate (e.g. sodium alginate), a cellulose and cellulose derivatives (e.g. carboxymethylcellulose, hydroxypropyl cellulose, hydroxyethyl cellulose, microcrystalline cellulose, a combination thereof, and the like), chitosan, gelling proteins (e.g. gelatin), hydroxyethyl methacrylate, a modified starch (e.g. thermally modified starch, and the like), natural gums and polysaccharides (e.g. Acacia, gum Arabic, Guar gum, gum Karaya, pectin, tragacanth, a combination thereof, and the like), a polyacrylic acid (e.g. CARBOPOL®934P from Lubrizol CAS no. 9003-01-4), a poly(acrylic acid/divinyl benzene), a poly(lactic acid), a polycarbophil (i.e. polyacrylic acid cross-linked with divinyl glycol), polyvinyl pyrrolidone, psyllium, a resin (e.g. Amberlite-200 a cation exchange resin based on sulfonic acid exchange groups on a polystyrenic matrix, and the like), or a combination thereof. Specific mucoadhesive agents include microcrystalline cellulose, carboxymethylcellulose sodium, polyvinyl pyrrolidone, or a combination thereof.

The composition can optionally further comprise an intranasal formulation excipient such as a buffering agent, a flavoring agent, a sweetening agent, a tonicity agent, an antimicrobial preservative, an antimicrobial preservative synergist, a surfactant, an emulsifier, a solubilizer, an absorption enhancer, or a combination thereof. In some instances, a single compound or material will meet two or more of the foregoing general classifications. For example, a compound may function as both an emulsifier and a surfactant.

The liquid mucoadhesive intranasal dosage composition can further include an antimicrobial preservative to prevent the unwanted growth of bacteria, molds, fungi, or yeast. Examples of suitable antimicrobial preservatives include benzyl alcohol, benzalkonium chloride, benzoic acid alkali metal salts (e.g., sodium benzoate), sorbic acid alkali metal salts (e.g., potassium sorbate), sodium erythorbate, sodium nitrite, calcium sorbate, butylated hydroxyanisole (BHA), butylated hydroxytoluene (BHT), parabens (e.g., lower alkyl esters of para-hydroxybenzoic acid), alkali metal salts of parabens including sodium and potassium salts of methyl-, ethyl-, propyl-, or butylparaben, or a combination thereof. Specific antimicrobial preservatives include benzyl alcohol, benzalkonium chloride, or a combination thereof.

The antimicrobial preservative can be present in the liquid mucoadhesive intranasal dosage composition in an amount of about 0.001 to about 1.0% w/v, specifically about 0.01 to about 0.55% w/v, and yet more specifically about 0.1 to about 0.3% w/v.

The liquid mucoadhesive intranasal dosage composition can further optionally include an antimicrobial preservative synergist such as ethylenediaminetetraacetic acid (EDTA) or pharmaceutically acceptable salts there of (e.g. calcium disodium EDTA). The antimicrobial preservative synergist can be present in the formulation in an amount of about 0.001 to about 0.1% w/v, specifically about 0.01 to about 0.05% w/v, and more specifically about 0.02 to about 0.04% w/v.

The liquid mucoadhesive intranasal dosage composition can be isotonic or isotonic and buffered. The liquid mucoadhesive intranasal dosage formulation may optionally comprise a tonicity agent such as dextrose, glycerin, mannitol, potassium chloride, sodium chloride, or a combination thereof. The amount of tonicity agent can be determined by one having ordinary skill in the art without undue experimentation.

The liquid mucoadhesive intranasal dosage composition can comprise a buffering agent. Exemplary buffering agents include citrates, acetates, phosphates (e.g. citric acid, sodium citrate, sodium acetate, dibasic sodium phosphate, monobasic sodium phosphate, or a combination thereof).

In further embodiments, any of the variant lectin polypeptides or variant lectin polypeptide dimers disclosed herein can be formulated such that they are suitable for topical application or administration. Thus, the compositions described herein are stable, cosmetically elegant, and well tolerated on subjects. By way of non-limiting example, the compositions described herein can be formulated as a solution, suspension, gel, hydrogel, cream, emulsion, micro-emulsion, nano-emulsion, lotion, spray, ointment, patch, tissue cloth, wipe, soap, paste, aerosol, and mask suitable for topical use. The variant lectin polypeptides or variant lectin polypeptide dimers disclosed herein can be incorporated into these topical formulations in an amount between 0.01 w % to the limit of solubility.

### F. Feed and Feed Additive Formulations

Any of the variant lectin polypeptides or variant lectin polypeptide dimers disclosed herein, either alone or in combination with at least one direct fed microbial alone and/or in combination with least one enzyme may be encapsulated for use in animal feed or a premix. In addition, a variant lectin polypeptide or variant lectin polypeptide dimer disclosed herein, either alone or in combination with at least one direct fed microbial alone and/or in combination with least one protease, amylase, xylanase, beta-glucosidase, and/or phytase, whether or not encapsulated, may be in the form of a granule.

Animal feeds may include plant material such as corn, wheat, sorghum. soybean, canola, sunflower or mixtures of any of these plant materials or plant protein sources for poultry, pigs, ruminants, aquaculture and pets. The terms “animal feed,” “feedstuff’ and “fodder” are used interchangeably and can comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grains with Solubles (DDGS) (particularly corn based Distillers Dried Grains with Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; and/or e) minerals and vitamins.

When used as, or in the preparation of, a feed, such as functional feed, the variant lectin or variant lectin-containing feed additive composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient. For example, there could be mentioned at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben. In some embodiments, the variant lectin or variant lectin-containing feed additive composition is fused to a carrier molecule, such as any of those disclosed herein.

In one embodiment the variant lectin or variant lectin-containing composition of the present invention is admixed with a feed component to form a feedstuff. The term “feed component” as used herein means all or part of the feedstuff. Part of the feedstuff may mean one constituent of the feedstuff or more than one constituent of the feedstuff, e.g. 2 or 3 or 4 or more. In one embodiment the term “feed component” encompasses a premix or premix constituents.

Preferably, the feed may be a fodder, or a premix thereof, a compound feed, or a premix thereof. A feed additive composition according to the present invention may be admixed with a compound feed, a compound feed component or to a premix of a compound feed or to a fodder, a fodder component, or a premix of a fodder. The term “fodder” as used herein means any food which is provided to an animal (rather than the animal having to forage for it themselves). Fodder encompasses plants that have been cut. Furthermore, fodder includes silage, compressed and pelleted feeds, oils and mixed rations, and also sprouted grains and legumes. Fodder may be obtained from one or more of the plants selected from: corn (maize), alfalfa (Lucerne), barley, birdsfoot trefoil, brassicas, Chau moellier, kale, rapeseed (canola), rutabaga (swede), turnip, clover, alsike clover, red clover, subterranean clover, white clover, fescue, brome, millet, oats, sorghum, soybeans, trees (pollard tree shoots for tree-hay), wheat, and legumes.

The term “compound feed” means a commercial feed in the form of a meal, a pellet, nuts, cake or a crumble. Compound feeds may be blended from various raw materials and additives. These blends are formulated according to the specific requirements of the target animal. Compound feeds can be complete feeds that provide all the daily required nutrients, concentrates that provide a part of the ration (protein, energy) or supplements that only provide additional micronutrients, such as minerals and vitamins. The main ingredients used in compound feed are the feed grains, which include corn, wheat, canola meal, rapeseed meal, lupin, soybeans, sorghum, oats, and barley.

Suitably a “premix” as referred to herein may be a composition composed of microingredients such as vitamins, minerals, chemical preservatives, antibiotics, fermentation products, and other essential ingredients. Premixes are usually compositions suitable for blending into commercial rations.

As used herein the term “contacted” refers to the indirect or direct application of a variant lectin or variant lectin-containing (or composition comprising a variant lectin polypeptide or variant lectin polypeptide dimer as described herein) to a product (e.g. the feed). Examples of application methods which may be used, include, but are not limited to, treating the product in a material comprising the feed additive composition, direct application by mixing the feed additive composition with the product, spraying the feed additive composition onto the product surface or dipping the product into a preparation of the feed additive composition. In one embodiment the feed additive composition of the present invention is preferably admixed with the product (e.g. feedstuff). Alternatively, the feed additive composition may be included in the emulsion or raw ingredients of a feedstuff.

It is also possible that the variant lectin polypeptide or variant lectin polypeptide dimer as described herein can be homogenized to produce a powder. In an alternative embodiment, a variant lectin polypeptide or variant lectin polypeptide dimer as described herein (or composition comprising a variant lectin polypeptide or variant lectin polypeptide dimer as described herein) can be formulated to granules as described in (referred to as TPT granules) or WO1997/016076 or WO1992/012645 incorporated herein by reference. “TPT” means Thermo Protection Technology. In another aspect, when the feed additive composition is formulated into granules the granules comprise a hydrated barrier salt coated over the protein core. The advantage of such salt coating is improved thermo-tolerance, improved storage stability and protection against other feed additives otherwise having adverse effect on the enzyme. Preferably, the salt used for the salt coating has a water activity greater than 0.25 or constant humidity greater than 60% at 20 C. In some embodiments, the salt coating comprises Na₂SO₄.

A method of preparing a variant lectin polypeptide or variant lectin polypeptide dimer as described herein may also comprise the further step of pelleting the powder. The powder may be mixed with other components known in the art. The powder, or mixture comprising the powder, may be forced through a die and the resulting strands are cut into suitable pellets of variable length.

Optionally, the pelleting step may include a steam treatment, or conditioning stage, prior to formation of the pellets. The mixture comprising the powder may be placed in a conditioner, e.g. a mixer with steam injection. The mixture is heated in the conditioner up to a specified temperature, such as from 60-100 C, typical temperatures would be 70 C, 80 C, 85 C, 90 C or 95 C. The residence time can be variable from seconds to minutes and even hours. Such as 5 seconds, 10 seconds, 15 seconds, 30 seconds, 1 minutes 2 minutes, 5 minutes, 10 minutes, 15 minutes, 30 minutes and 1 hour. It will be understood that a variant lectin polypeptide or variant lectin polypeptide dimer as described herein is suitable for addition to any appropriate feed material.

It will be understood by the skilled person that different animals require different feedstuffs, and even the same animal may require different feedstuffs, depending upon the purpose for which the animal is reared. Optionally, the feedstuff may also contain additional minerals such as, for example, calcium and/or additional vitamins. In some embodiments, the feedstuff is a corn soybean meal mix.

Feedstuff is typically produced in feed mills in which raw materials are first ground to a suitable particle size and then mixed with appropriate additives. The feedstuff may then be produced as a mash or pellets; the later typically involves a method by which the temperature is raised to a target level and then the feed is passed through a die to produce pellets of a particular size. The pellets are allowed to cool. Subsequently liquid additives such as fat and enzyme may be added. Production of feedstuff may also involve an additional step that includes extrusion or expansion prior to pelleting, in particular by suitable techniques that may include at least the use of steam.

The feedstuff may be a feedstuff for a monogastric animal, such as poultry (for example, broiler, layer, broiler breeders, turkey, duck, geese, water fowl), and swine (all age categories), a ruminant such as cattle (e.g. cows or bulls (including calves)), horses, sheep, a pet (for example dogs, cats) or fish (for example agastric fish, gastric fish, freshwater fish such as salmon, cod, trout and carp, e.g. koi carp, marine fish such as sea bass, and crustaceans such as shrimps, mussels and scallops).

The feed additive composition and/or the feedstuff comprising the same may be used in any suitable form. The feed additive composition may be used in the form of solid or liquid preparations or alternatives thereof Examples of solid preparations include powders, pastes, boluses, capsules, pellets, tablets, dusts, and granules which may be wettable, spray-dried or freeze-dried. Examples of liquid preparations include, but are not limited to, aqueous, organic or aqueous-organic solutions, suspensions and emulsions. In some applications, the feed additive compositions may be mixed with feed or administered in the drinking water. A feed additive composition, comprising admixing a variant lectin polypeptide or variant lectin polypeptide dimer as described herein with a feed acceptable carrier, diluent or excipient, and (optionally) packaging.

The feedstuff and/or feed additive composition may be combined with at least one mineral and/or at least one vitamin. The compositions thus derived may be referred to herein as a premix. The feedstuff may comprise at least 0.0001% by weight of the feed additive. Suitably, the feedstuff may comprise at least 0.0005%; at least 0.0010%; at least 0.0020%; at least 0.0025%; at least 0.0050%; at least 0.0100%; at least 0.020%; at least 0.100% at least 0.200%; at least 0.250%; at least 0.500% by weight of the feed additive.

Preferably, a food or feed additive composition may further comprise at least one physiologically acceptable carrier. The physiologically acceptable carrier is preferably selected from at least one of maltodextrin, limestone (calcium carbonate), cyclodextrin, wheat or a wheat component, sucrose, starch, Na₂SO₄, Talc, PVA and mixtures thereof. In a further embodiment, the food or feed additive may further comprise a metal ion chelator. The metal ion chelator may be selected from EDTA or citric acid.

Dry powder or granules may be prepared by means known to those skilled in the art, such as, high shear granulation, drum granulation, extrusion, spheronization, fluidized bed agglomeration, fluidized bed spray drying.

### III. Methods

### A. Cell Culture Methods

In certain embodiments, the present disclosure provides recombinant cells capable of producing proteins of interest (for example, a variant lectin polypeptide or variant lectin polypeptide dimer as described herein). More particularly, certain embodiments are related genetically modified (recombinant) host cells expressing heterologous variant lectin polypeptides or variant lectin polypeptide dimers as described herein. Thus, particular embodiments are related to cultivating (fermenting) host cells for the production of variant lectin proteins. In general, fermentation methods well known in the art are used to ferment the host cells.

In some embodiments, the cells are grown under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within batch cultures, cells progress through a static lag phase to a high growth log phase and finally to a stationary phase, where growth rate is diminished or halted. If untreated, cells in the stationary phase eventually die. In general, cells in log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Culturing/fermenting is generally accomplished in a growth medium comprising an aqueous mineral salts medium, organic growth factors, a carbon and energy source material, molecular oxygen, and, of course, a starting inoculum of the microbial host to be employed.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process, and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and also present preferably should be certain trace elements such as copper, manganese, molybdenum, zinc, iron, boron, and iodine, and others, again in suitable soluble assimilable form, all as known in the art.

The fermentation reaction is an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion.

The fermentation temperature can vary somewhat, but for most host cells the temperature generally will be within the range of about 20° C. to 40° C.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations and can be employed by bubbling through the aqueous ferment (fermentation medium) in suitable amounts. At the same time, such ammonia can also be employed to assist in pH control.

The pH range in the aqueous microbial ferment (fermentation admixture) should be in the exemplary range of about 2.0 to 8.0. Preferences for pH range of microorganisms are dependent on the media employed to some extent, as well as the particular microorganism, and thus change somewhat with change in media as can be readily determined by those skilled in the art.

In certain aspects, the fermentation is conducted in such a manner that the carbon-containing substrate can be controlled as a limiting factor, thereby providing good conversion of the carbon-containing substrate to cells and avoiding contamination of the cells with a substantial amount of unconverted substrate. The latter is not a problem with water-soluble substrates, since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

If desired, part or all of the carbon and energy source material and/or part of the assimilable nitrogen source such as ammonia can be added to the aqueous mineral medium prior to feeding the aqueous mineral medium to the fermenter.

Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermenter, cell density measurable by dry cell weights, light transmittancy, or the like. The feed rates of the various materials can be varied so as to obtain as rapid a cell growth rate as possible, consistent with efficient utilization of the carbon and energy source, to obtain as high a yield of microorganism cells relative to substrate charge as possible.

In either a batch, or the preferred fed batch operation, all equipment, reactor, or fermentation means, vessel or container, piping, attendant circulating or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermenter employed is not critical.

### B. Protein Recovery

The instant disclosure further describes and exemplifies particularly suitable processes (methods) for harvesting, clarifying, recovering, purifying and the like fermentation broths in which one or more variant lectin polypeptides or variant lectin polypeptide dimer proteins have been produced. Thus, certain embodiments are related to, inter alia, collecting broths at the end of fermentation, harvesting collected broths, recovering one or more variant lectin polypeptides or variant lectin polypeptide dimer proteins from a harvested broth (e.g., such as clarifying harvested broths, concentrating clarified broths, purifying clarified broth concentrates, etc.). In certain aspects, purified protein preparations are derived from fermentation broths collected and harvested as described herein.

Certain other aspects of the disclosure provide, inter alia, novel methods for the recovering and optionally purifying recombinantly-produced proteins (such as variant lectin polypeptides or variant lectin polypeptide dimer proteins) obtained from a recombinant cells expressing a recombinantly-produced protein (e.g., a recombinant Gram-negative cell, a recombinant Gram-positive cell, a recombinant a plant (e.g., tobacco) cell, a recombinant fungal cell, etc.). Certain other aspects of the disclosure provide, inter alia, novel methods for the recovery and optional purification of a variant lectin or variant lectin dimer obtained from naturally occurring sources.

Thus, in certain aspects, a variant lectin polypeptide or variant lectin polypeptide dimer protein preparation is recovered according to the compositions and methods of the disclosure. In other aspects, a variant lectin polypeptide or variant lectin polypeptide dimer preparation is recovered and purified according to the methods of the disclosure. As used herein, the terms “purified”, “isolated” or “enriched” with regard to a protein means that the variant lectin polypeptide or variant lectin polypeptide dimer is transformed from a less pure state by virtue of separating it from some, or all of, the contaminants with which it is associated. Contaminants include, but are not limited to, microbial cells, metabolites, solvents, chemicals, color, inactive forms of the target variant lectin or variant lectin dimer, aggregates, process aids, inhibitors, fermentation media, cell debris, nucleic acids, proteins other than the target variant lectin or variant lectin dimer protein, host cell proteins, cross-contaminants from the production equipment and the like.

Thus, in the context of a “purified variant lectin polypeptide or variant lectin polypeptide dimer” as used herein, purification may be accomplished by any art-recognized separation techniques, including, but not limited to, ion exchange chromatography, affinity chromatography, hydrophobic separation, dialysis, protease treatment, heat treatment. ammonium sulphate precipitation or other protein salt precipitation, crystallization, centrifugation, size exclusion chromatography, filtration, microfiltration, gel electrophoresis, or separation on a gradient to remove whole cells, cell debris, impurities, extraneous proteins, or enzymes undesired in the final composition.

It is further possible to then add constituents to a purified or isolated variant lectin polypeptide or variant lectin polypeptide dimer composition which provide additional benefits, for example, activating agents, anti-inhibition agents, desirable ions, compounds to control pH or other enzymes or chemicals.

As used herein, variant lectin “purity” is a relative term, and is not meant to be limiting, when used in phrases such as a “recovered variant lectin is of higher purity, the same purity, or lower purity than prior to the recovery process”. For example, the relative “purity” of a protein, before and after a recovery process, may be determined using methods known in the art, including but not limited to, general quantification methods (e.g., Bradford, UV-Vis, activity assays), electrophoretic analysis (SDS-PAGE), analytical HPLC, mass spectrometry, hydrophobic interaction chromatography and the like.

Thus, according to certain aspects, variant lectin polypeptide or variant lectin polypeptide dimer protein preparations are recovered from fermentation broths, wherein the recovered variant lectin preparations are of higher purity after performing one or more recovery processes described herein. For example, a fermentation broth (e.g., a whole broth at the end of fermentation) may be subjected to one or more protein recovery processes including, but not limited to, broth conditioning processes, broth clarification processes, protein enrichment and/or protein purification processes (e.g., protein concentration, filtration, precipitation, crystallization, crystal separation, crystal sludge dissolution processes and the like), buffer exchange processes, sterile filtration processes and the like. In certain aspects, the fermentation broth is subjected to a broth treatment (broth conditioning) process to improve subsequent broth handling properties.

In certain embodiments, such as when a host cell has been constructed for intracellular variant lectin of variant lectin dimer expression, a fermentation broth is subjected to a cell lysis process. For example, cell lysis processes include without limitation, enzymatic treatments (e.g., lysozyme, proteinase K treatments), chemical means (e.g., ionic liquids), physical means (e.g., French pressing, ultrasonic), simply holding culture without feeds, and the like. Thus, in certain preferred embodiments, a broth lysis process releases variant lectin into the (lysed) cell broth.

Thus, as described herein, the methods/processes of the disclosure are not meant to be limiting, as one of skill may readily adapt or modify one or more of the compositions and/or methods disclosed herein for the recovery of specific variant lectin of variant lectin dimer proteins, and/or combinations thereof.

### C. Methods for Treating or Preventing Viral Infection

Also provided herein are methods for treating or preventing a viral infection in an animal in need thereof by administering one or more of the variant lectin polypeptides or variant lectin polypeptide dimers disclosed herein or compositions containing one or more of the variant lectin polypeptides or variant lectin polypeptide dimers to the animal.

In one embodiment, the variant lectin proteins or functional fragment thereof comprises an amino acid sequence at least about 60% identical (such as any of about 60%, 61%, 62%, 63%, 64%, 65%, 66%, 67%, 68%, 69%, 70%, 71%, 72%, 73%, 74%, 75%, 76%, 77%%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 or SEQ ID NO:18, wherein the variant lectin comprises one or more (such as 1, 2, 3, 4, or 5) substitutions at positions 10, 38, 53, 54. and 78. In some embodiments, the variant lectin polypeptide is administered to the animal (such as, for example, a swine or a horse) via oral, nasal, and/or topical administration. The variant lectin proteins or functional fragment thereof can exhibit anti-viral activity against infection with one or more of porcine reproductive and respiratory syndrome (PRRSV), porcine epidemic diarrhea virus (PEDV), porcine rotavirus, or equine viral arteritis (EVA).

In another embodiment, variant lectin polypeptide dimers or functional fragment thereof comprises two amino acid sequences at least about 60% identical (such as any of about 60%, 61%, 62%, 63%, 64%, 65%, 66%, 67%, 68%, 69%, 70%, 71%, 72%, 73%, 74%, 75%, 76%, 77%%. 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to SEQ ID NO:8 and/or SEQ ID NO:18, wherein each variant lectin component of the dimer comprises one or more (such as 1, 2, 3, 4, or 5) substitutions at positions 10, 38, 53, 54, and 78. Each variant lectin component of the dimer can be identical or can possess unique substitutions relative to the other member in separate embodiments. In some embodiments, the variant lectin polypeptide is administered to the animal (such as, for example, a swine or a horse) via oral, nasal, and/or topical administration. The variant lectin polypeptide dimers or functional fragment thereof can exhibit anti-viral activity against infection with one or more of porcine reproductive and respiratory syndrome (PRRSV), porcine epidemic diarrhea virus (PEDV), porcine rotavirus, or equine viral arteritis (EVA).

The invention can be further understood by reference to the following examples, which are provided by way of illustration and are not meant to be limiting.

### EXAMPLES

### Example 1: Engineering Griffithsin for One or More of Improvement in Expression, Stability, Solubility, Isoelectric Point or Binding

This example describes the creation of a library for engineering Griffithsin for one or more of improvement in expression, stability, solubility, isoelectric point or binding.

Library creation: Positions 10, 31, 36, 38, 48, 49, 53, 54, 78, and 83, based on the sequence of SEQ ID NO:8, were chosen for saturation library creation. Eight of these positions have never been engineered, and at position 78 only the substitution of the native M to Q has been thoroughly investigated. There may be other substitutions that work as well or better than Q at this position. In addition, position 31 is occupied by a non-natural amino acid in the native algae produced protein. This has been replaced by Alanine in the recombinant versions of Griffithsin, but other replacement amino acids may be additionally be preferable.

The Griffithsin library was constructed as follows. A first DNA fragment comprising a (5′) aprE gene flanking region (5′ aprE gene FR) which includes a Bacillus subtilis aprE homology (SEQ ID NO: 16) was operably linked to a polynucleotide construct comprising B. subtilis rrnI-p2 promoter region DNA sequence (SEQ ID NO: 2; SEQ ID NO:40 from WO2020112609) operably linked to a DNA sequence of the B. subtilis aprE 5′ untranslated region (5′-UTR; SEQ ID NO: 3) operably linked to DNA encoding the B. subtilis AprE signal sequence (SEQ ID NO: 4) operably linked to a DNA sequence encoding the mature Griffithsia sp. variant sequences operably linked to a B. amyloliquefaciens Bpn′ terminator (SEQ ID NO: 6) operably linked to a selectable marker (SEQ ID NO: 5) operably linked to a (3′) aprE gene flanking region (3′ aprE gene FR) (SEQ ID NO: 15).

More particularly, these DNA fragments were assembled using standard molecular biology techniques and were used as a template to develop linear DNA expression cassettes comprising one or more mature sequence modifications described herein. For example, B. subtilis strains comprising variant mature region sequences were constructed by integrating the DNA fragments (described above) in the genome. where the fragments contain the mature sequence variants. The variant expression cassettes were developed as 5.7 kb linear DNA fragment and used to transform competent B. subtilis cells, wherein the transformation mixtures were plated onto LA plates containing 5 ppm kanamycin and incubated overnight at 37° C. Single colonies were picked and grown in Luria broth at 37° C. under antibiotic selection.

For variant assay experiments, transformed cells were grown in 96-well MTPs in a semi-defined cultivation medium (enriched media based on MOPS buffer) for 2 days at 37° C., 270 rpm, with 80% humidity in shaking incubator, which were then harvested.

Construction of Griffithsin M78 linked dimers (GGG, GTG, GPG): Construction of linked dimers of Griffithsin M78 (SEQ ID NO:7) was performed by overlapping PCR using oligonucleotides to incorporate the linker GGG or GTG or GPG resulting in the dimers nucleotide sequences (SEQ ID NOs: 19, 28, 30).

A first DNA fragment comprising a (5′) aprE gene flanking region (5′ aprE gene FR) which includes a Bacillus subtilis aprE homology (SEQ ID NO: 16) was operably linked to a polynucleotide construct comprising B. subtilis rrnI-p2 promoter region DNA sequence (SEQ ID NO: 2), operably linked to a DNA sequence of the B. subtilis aprE 5′ untranslated region (5′-UTR; SEQ ID NO: 3), in some instances operably linked to DNA encoding the B. subtilis AprE signal sequence (SEQ ID NO: 4), operably linked to the DNA sequence encoding the mature Griffithsia sp. M78 dimer sequence (SEQ ID NO: 19 or 28 or 30), operably linked to a B. amyloliquefaciens Bpn′ terminator (SEQ ID NO: 6), operably linked to a selectable marker (SEQ ID NO: 29), operably linked to a (3′) aprE gene flanking region (3′ aprE gene FR) (SEQ ID NO: 15).

These DNA fragments described above were assembled using standard molecular biology techniques. The dimer sequences were constructed as 6 kb linear DNA fragment expression cassettes which were then used to transform into competent B. subtilis nine proteases deleted cells. The nine proteases deleted in this strain are aprE, nprE, epr, ispA, bpr, mpr, vpr, wprA and nprB. The transformation mixtures were plated onto LA plates selective for prototrophy and incubated overnight at 37° C. Single colonies were picked and grown in Luria broth at 37° C. under prototrophic selection. Transformed cells were grown in 24-well MTPs in cultivation medium (enriched semi-defined media based on MOPS buffer, with urea as major nitrogen source, maltodextrin as the main carbon source, supplemented with 3% soytone for robust cell growth, containing antibiotic selection) for 48 hours at 37° C., 250 rpm, with 80% humidity in shaking incubator. Intracellular samples were analyzed by SDS-PAGE gel.

Construction of the Griffithsin variant S10N/G053K/M78Y linked dimers: A library of linkers connecting two monomers of Griffithsin variant S10N/G053K/M78Y (SEQ ID NO. 13) was designed and ordered synthetically. The library includes 27 linker sequences (SEQ ID NO: 32 to 57). Construction of the strains expressing the dimers of the Griffithsin variant sequence (SEQ ID NO. 13) was carried on similarly as described above.

These DNA fragments described above were assembled using standard molecular biology techniques. The dimer sequences were constructed as 6 kb linear DNA fragment expression cassettes which were then used to transform into competent B. subtilis five proteases deleted cells. The five proteases deleted in this strain are aprE, nprE, epr, ispA, bpr. Transformed cells were grown in 24-well MTPs in cultivation medium (enriched semi-defined media based on MOPS buffer, with urea as major nitrogen source, maltodextrin as the main carbon source, supplemented with 3% soytone for robust cell growth, containing antibiotic selection) for 48 hours at 37° C., 250 rpm. with 80% humidity in shaking incubator. Intracellular samples were analyzed by SDS-PAGE gel.

### Example 2: Measurement of Performance Index (PI) and Thermal Melting Point of Griffithsin Variants

Measurement of library variants expression using UPLC: To generate the Griffithsin samples for screening, the transformed B. subtilis cells were grown in 96 well microtiter plates (MTPs, manufacture) at 37° C. for 68 hours in a semi-defined cultivation medium. 30 ul of 3N H2SO4 was added into each well (350 ul) so that the resulting culture broth is at pH2. Cultures were harvested by centrifugation at 3000 g for 15 min and filtered through Multiscreen® filter plates (EMD Millipore, Billerica, MA, USA). The filtered culture supernatants were used for the assays described below.

Typically, 280 μl of filtered culture broth was neutralized by adding 150 μl of 1.5N Sodium Citrate at pH9 so that the resulting pH to be pH6.5. The neutralized sample was further diluted 5-fold in MilliQ H₂O in a new 96 well plate (NUNC, 267245). 10 μl of this diluted sample was loaded onto UPLC for quantitation. Griffithsin concentration was determined by separation of protein components using a Zorbax 300 SB-C3 column (Agilent) and running a linear gradient of 0.1% Trifluoroacetic acid in water (Buffer A) and 0.1% Trifluoroacetic acid in Acetonitrile (Buffer B) with detection at 220 nm column on UHPLC. The Griffithsin concentration of the samples was calculated using a standard curve of the purified reference wild type Griffithsin. Expression performance index (PI) was calculated by dividing the variant expression over N-GRFT (M078N) expression. The PT expression of variants are summarized in Table 1.

Measurement of thermal melting point of Griffithsin variants using Sypro orange dye binding: Fifteen μl of neutralized MTP Griffithsin sample was mixed with 5 μl of 125 fold diluted sypro orange (Thermo Fisher, S6650), they were mixed in Roche 384 well qPCR plate (Thermo Fisher, 4309849). The plate was sealed with Roche seal and quickly spun down. Roche 480 light cycler was used to measure the Tm. The fluorescent change was recorded at a scan rate of ˜1C/min, the data was exported and was processed using R scripts. Transitions corresponding to Griffithsin thermal melting temperature was compared. The PI of Tm of variants are calculated by subtracting N-GRFT (M078N) Tm from each variant's Tm value. This PI Tm of all tested variants are listed in the Table 1.

TABLE 1
Performance index (PI) of Tm and expression relative
to M078N are listed for all variants tested. Sequence
numbering is based on that of SEQ ID No: 8.

| | Tm | PI |
|---|---|---|
| Mutation | PI | Expression |
| A031C-M078N | −3.02 | 0.748 |
| A031G-M078N | −3.08 | 0.705 |
| A031S-M078N | −5.19 | 1.281 |
| G036A-M078N | −4.87 | 0.826 |
| G036C-M078N | −3.67 | 0.451 |
| G036E-M078N | −4.87 | 0.865 |
| G036F-M078N | −1.6 | 0.133 |
| G036H-M078N | −3.5 | 0.656 |
| G036K-M078N | −4.41 | 0.705 |
| G036L-M078N | −1.97 | 0.128 |
| G036M-M078N | −5.09 | 0.495 |
| G036N-M078N | −2.63 | 0.838 |
| G036Q-M078N | −4.42 | 0.666 |
| G036R-M078N | −4.58 | 0.570 |
| G036S-M078N | −4.07 | 0.897 |
| G036T-M078N | −5.63 | 0.665 |
| G036W-M078N | −5.49 | 0.267 |
| G036Y-M078N | −3.64 | 0.529 |
| G053A-M078N | 0.6 | 1.115 |
| G053C-M078N | 1.06 | 0.515 |
| G053D-M078N | 0.5 | 0.895 |
| G053E-M078N | 1.19 | 1.356 |
| G053F-M078N | −0.32 | 0.894 |
| G053H-M078N | 0.47 | 1.000 |
| G053I-M078N | −0.05 | 0.588 |
| G053K-M078N | 0.76 | 1.276 |
| G053L-M078N | 0.38 | 0.847 |
| G053N-M078N | −0.22 | 0.930 |
| G053Q-M078N | 0.9 | 1.253 |
| G053R-M078N | 0.65 | 0.974 |
| G053S-M078N | 0.13 | 0.988 |
| G053V-M078N | 0.42 | 0.790 |
| G053W-M078N | 0.16 | 0.758 |
| G053Y-M078N | 0.15 | 0.909 |
| H038A-M078N | 0.01 | 0.999 |
| H038C-M078N | 0.62 | 0.534 |
| H038D-M078N | −2.89 | 1.162 |
| H038E-M078N | −0.15 | 1.150 |
| H038F-M078N | −2.9 | 0.985 |
| H038G-M078N | −2.73 | 0.893 |
| H038I-M078N | 0.2 | 1.111 |
| H038K-M078N | −0.09 | 1.059 |
| H038L-M078N | 0.14 | 0.947 |
| H038M-M078N | −0.17 | 1.113 |
| H038N-M078N | −1.18 | 0.982 |
| H038P-M078N | 0.59 | 0.934 |
| H038Q-M078N | 0.91 | 0.934 |
| H038R-M078N | −0.09 | 0.986 |
| H038S-M078N | −0.13 | 0.915 |
| H038T-M078N | −0.57 | 1.109 |
| H038V-M078N | 0.02 | 1.163 |
| H038W-M078N | −3.03 | 0.855 |
| H038Y-M078N | −2.88 | 1.057 |
| M078W | 2.8 | 1.413 |
| M078C | 1.44 | 0.780 |
| M078D | −2.41 | 0.723 |
| M078E | 0.01 | 1.195 |
| M078H | 1.74 | 1.023 |
| M078S | −0.08 | 1.026 |
| M078T | 0.58 | 1.157 |
| M078Y | 1.76 | 1.313 |
| P048A-M078N | −2.15 | 0.668 |
| P048C-M078N | 0.84 | 0.081 |
| P048D-M078N | −2.89 | 1.165 |
| P048E-M078N | −0.61 | 1.324 |
| P048H-M078N | −3.21 | 1.123 |
| P048K-M078N | −2.66 | 1.170 |
| P048L-M078N | −2.76 | 0.522 |
| P048N-M078N | −3.36 | 0.935 |
| P048Q-M078N | −3.1 | 1.043 |
| P048R-M078N | −2.77 | 1.124 |
| P048S-M078N | −3.24 | 1.167 |
| P048T-M078N | −2.77 | 0.859 |
| P048W-M078N | −3.41 | 0.137 |
| P048Y-M078N | −2.76 | 0.253 |
| S010I-M078N | −0.61 | 0.831 |
| S010P-M078N | 0.54 | 0.988 |
| S054A-M078N | −0.12 | 0.785 |
| S054D-M078N | −0.65 | 0.804 |
| S054E-M078N | −0.55 | 0.844 |
| S054F-M078N | −2.15 | 0.909 |
| S054G-M078N | −1.39 | 0.998 |
| S054H-M078N | −1.62 | 1.018 |
| S054I-M078N | −0.85 | 0.770 |
| S054K-M078N | −1.03 | 1.107 |
| S054L-M078N | −1.62 | 0.880 |
| S054M-M078N | −0.95 | 0.962 |
| S054N-M078N | −0.92 | 0.857 |
| S054P-M078N | 2.7 | 0.306 |
| S054Q-M078N | −0.05 | 0.927 |
| S054R-M078N | −1.13 | 0.959 |
| S054T-M078N | 1.89 | 0.234 |
| S054V-M078N | −0.49 | 1.062 |
| S054W-M078N | −3.8 | 0.666 |
| S054Y-M078N | −2.41 | 0.887 |
| T049A-M078N | −3.54 | 0.890 |
| T049C-M078N | −3.42 | 0.510 |
| T049D-M078N | −5.03 | 0.653 |
| T049E-M078N | −2.59 | 0.797 |
| T049F-M078N | −5.18 | 0.718 |
| T049G-M078N | −6.24 | 0.636 |
| T049H-M078N | −5.44 | 0.756 |
| T049K-M078N | −3.29 | 0.950 |
| T049L-M078N | −5.4 | 0.403 |
| T049M-M078N | −3.81 | 0.854 |
| T049N-M078N | −4.06 | 0.646 |
| T049P-M078N | −4.3 | 0.622 |
| T049Q-M078N | −3.27 | 0.802 |
| T049R-M078N | −4.05 | 0.860 |
| T049S-M078N | −3.48 | 0.992 |
| T049V-M078N | −1.75 | 0.992 |
| T049W-M078N | −7.01 | 0.565 |
| T049Y-M078N | −5.37 | 0.728 |
| G036D | −2.9 | 0.997 |
| G036F | −3.4 | 0.610 |
| G036L | −1.36 | 0.283 |
| G036W | −4.73 | 0.594 |
| G053A | 2.11 | 1.783 |
| G053C | 2.31 | 0.968 |
| G053D | 1.99 | 0.954 |
| G053E | 2.05 | 1.672 |
| G053H | 1.32 | 1.331 |
| G053K | 2.77 | 1.981 |
| G053L | 2.76 | 1.794 |
| G053M | 2.43 | 1.827 |
| G053P | −4.18 | 0.737 |
| G053Q | 2.23 | 1.434 |
| G053R | 2.36 | 1.436 |
| G053T | 1.96 | 1.221 |
| G053V | 2.08 | 1.528 |
| G083P | −4.82 | 0.225 |
| G083T | 0.08 | 0.036 |
| H038C | 1.81 | 0.459 |
| H038P | 2.02 | 1.177 |
| H038Q | 2.03 | 1.451 |
| P048C | −2.34 | 1.491 |
| P048F | −0.57 | 0.567 |
| P048G | −2.73 | 1.052 |
| P048M | 3.19 | 0.080 |
| P048W | 2.72 | 0.041 |
| P048Y | −1.94 | 0.078 |
| S010A | 0.63 | 1.045 |
| S010C | 2 | 0.243 |
| S010D | 2 | 1.099 |
| S010E | 1.39 | 1.217 |
| S010F | 0.32 | 0.720 |
| S010G | 1.33 | 0.918 |
| S010H | 0.98 | 1.109 |
| S010K | 2.1 | 0.538 |
| S010L | 0.15 | 0.668 |
| S010M | 0.7 | 0.978 |
| S010N | 1.43 | 1.247 |
| S010P | 2 | 1.296 |
| S010Q | 1.15 | 1.325 |
| S010T | 1.57 | 1.351 |
| S010V | 0.65 | 1.011 |
| S010W | −0.29 | 0.511 |
| S010Y | 0.21 | 0.934 |
| S054C | −0.09 | 0.643 |
| S054P | 1.54 | 1.493 |
| S054T | 1.34 | 1.380 |
| T049I | −1.68 | 1.285 |
| S010N-G053L-S054P-N078Q | −0.2 | 1.281 |
| S010D-G053E-S054P-N078Y | 2.28 | 0.187 |
| S010D-N078Q | 0.07 | 0.730 |
| S010N-H038Q-G053E-S054P-N078M | 1.73 | 1.521 |
| S010N-H038Q-G053E-N078M | 2.08 | 1.244 |
| S010N-H038Q-G053K-S054P-N078Y | 2.89 | 1.262 |
| S010N-H038Q-G053L-S054P-N078Q | 1.67 | 1.291 |
| S010D-H038Q-G053E-N078Q | 2.16 | 0.866 |
| H038Q-N078Q | 1.11 | 0.788 |
| S010D-G053E-S054P-N078M | 1.27 | 1.713 |
| S010D-H038Q-G053K-S054P-N078Y | 3.17 | 1.251 |
| G053K-N078Y | 2.32 | 0.945 |
| S010D-G053K-N078Q | 1.67 | 1.060 |
| S010N-G053E-N078Y | 1.23 | 1.549 |
| S010N-G053L-N078Q | −0.67 | 1.091 |
| S010N-G053L-S054P-N078M | 1.05 | 1.273 |
| S010N-G053E-N078Q | 0.16 | 1.370 |
| S010N-N078M | 0.19 | 0.864 |
| S010N-G053E-S054P-N078M | 0.68 | 1.117 |
| S010D-N078Y | 1.04 | 0.988 |
| S010N-G053L-S054P-N078Y | 2.13 | 1.541 |
| S010D-G053E-N078Q | 0.84 | 0.795 |
| S010N-H038Q-G053L-N078M | 2.95 | 1.019 |
| H038Q-N078M | 0.98 | 0.943 |
| S010D-H038Q-G053K-S054P-N078M | 2.48 | 1.653 |
| S010D-G053K-S054P-N078M | 1.38 | 1.324 |
| S010D-H038Q-G053L-N078Y | 3.35 | 1.558 |
| S010D-G053K-S054P-N078Y | 1.54 | 1.802 |
| S010N-H038Q-G053E-N078Y | 2.32 | 1.845 |
| S010D-H038Q-G053L-N078M | 2.34 | 1.036 |
| S010D-G053E-S054P-N078Q | 0.49 | 1.361 |
| S010N-G053L-N078Y | 2.21 | 1.195 |
| S010N-G053E-S054P-N078Q | 0.23 | 1.083 |
| S010N-G053K-N078M | 2.27 | 0.258 |
| S010D-H038Q-G053E-N078Y | 3.41 | 1.041 |
| S010D-H038Q-G053K-N078Y | 3.78 | 1.177 |
| S010D-H038Q-G053K-N078M | 2.97 | 1.045 |
| S010D-G053L-N078Y | 2.39 | 1.534 |
| N078Y | 0.14 | 1.146 |
| S010N-H038Q-G053K-S054P-N078Q | 2.27 | 0.352 |
| S010N-H038Q-G053K-N078Y | 2.53 | 1.191 |
| S010N-H038Q-G053L-N078Y | 2.93 | 1.284 |
| S054P-N078M | −0.09 | 1.153 |
| S010D-G053L-N078M | 1.4 | 0.919 |
| S010N-G053K-S054P-N078M | 1.34 | 1.089 |
| S010N-G053K-S054P-N078Q | 1.01 | 0.980 |
| G053E-N078Y | 1.32 | 1.113 |
| S010D-H038Q-G053L-S054P-N078Q | 2.29 | 1.248 |
| H038Q-S054P-N078M | 0.57 | 1.613 |
| S010N-H038Q-G053L-N078Q | 0.96 | 1.209 |
| S010N-H038Q-G053K-S054P-N078M | 2.62 | 0.320 |
| S010N-G053L-N078M | 0.66 | 1.275 |
| S010D-H038Q-G053E-N078M | 1.15 | 1.096 |
| S010N-G053K-N078Q | 0.97 | 0.794 |
| G053E-N078Q | −0.09 | 0.861 |
| S010D-H038Q-G053K-N078Q | 2.85 | 0.922 |
| S010N-H038Q-G053K-N078Q | 2.71 | 1.115 |
| S010D-G053K-S054P-N078Q | 1.67 | 1.026 |
| S010N-G053K-N078Y | 2.5 | 1.012 |
| G053K-N078Q | 1.67 | 0.847 |
| S010D-H038Q-G053L-N078Q | 0.97 | 0.720 |
| S010N-G053K-S054P-N078Y | 2.05 | 0.666 |
| S010N-G053E-N078M | 0.9 | 1.653 |
| S010D-G053L-N078Q | 0.68 | 1.063 |
| G053K-N078M | 1.45 | 0.857 |
| S010N-N078Y | 0.59 | 1.230 |
| S010N-H038Q-G053L-S054P-N078Y | 2.76 | 1.126 |
| G053L-N078Q | 0.3 | 0.584 |
| S010N-H038Q-G053K-N078M | 3.02 | 1.260 |
| H038Q-S054P-N078Q | 1.56 | 1.028 |
| S010N-H038Q-G053E-N078Q | 1.65 | 1.668 |
| S010N-G053E-S054P-N078Y | 0.93 | 1.861 |
| S010N-N078Q | 0.27 | 1.097 |
| S010D-H038Q-G053K-S054P-N078Q | 2.77 | 1.603 |
| S010D-G053K-N078Y | 2.39 | 1.913 |
| G053L-N078M | 0.96 | 1.589 |
| S010D-G053L-S054P-N078Q | 1.2 | 0.933 |

### Example 3: Assessment of Griffithsin Protease Stability

Selected variants were grown in shake flasks and tested for protease stability in the presence of the protease FNA (BPN′ Y217L variant subtilisin from B. amyloliquefaciens) or Pepsin. For FNA (in house purified enzyme) treatment, 10 ppm FNA was incubated with 100 ul filtered shake flask sample for 42 hours to 7 days at pH 6.5. At the end of incubation, samples were run on SDS-PAGE gel, Griffithsin band intensity was quantitated in comparison to no FNA treat controls. For pepsin treatment (Sigma, P7125-100 g), 8 ppm pepsin was incubated with 100 μl of crude Griffithsin sample at pH 2 for 22 hours to 7 days, at 22 hours or 7 days, samples were run on SDS-PAGE for Griffithsin quantitation. The band intensity relative to its control without FNA or Pepsin treatment was compared. The data was summarized in Table 2.

TABLE 2
Results of protease stability assay for selected variants

| | band ratio | band ratio |
|---|---|---|
| | after/before | after/before |
| Mut | FNA treatment | Pepsin treatment |
| N078Y | 1.07 | 0.85 |
| N078T | 1.04 | 0.77 |
| M078W | 1.06 | 0.81 |
| S010N | 1.21 | 0.87 |
| S010Q | 1.40 | 0.91 |
| S010D | 1.19 | 0.88 |
| S054P | 1.11 | 1.22 |
| G053R | 1.00 | 0.76 |
| G053E | 1.44 | 0.77 |
| G053A | 1.26 | 0.82 |
| G053K | 1.28 | 0.95 |
| G053Q | 1.38 | 0.83 |
| H038Q | 1.31 | 0.91 |
| S054T | 1.53 | 0.91 |
| G053L | 1.09 | 0.94 |

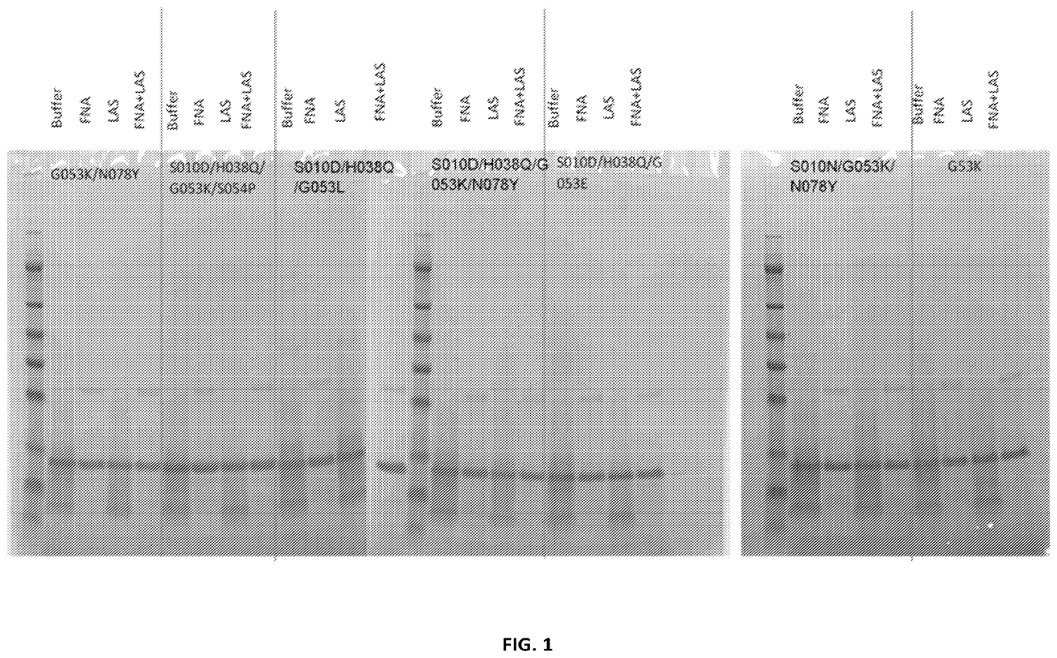

A set of combinatorial variants were grown in shake flasks. They were picked for comparison of their stability in the presence of protease FNA or LAS (LAS is Linear Alkylbenzene Sulfonate, a surfactant) or combination of FNA in LAS. For FNA or surfactant treatment, 10 ppm FNA in buffer or in 0.05% LAS or 0.05% LAS alone was incubated with 100 ul semi-purified sample for 5 days at pH6.5. At the end of incubation, samples were run on SDS-PAGE gel, in comparison to no treat controls. SDS-PAGE gel seen in FIG. 1.

### Example 4: Cytotoxicity Testing for Griffithsin Variants

103-104 cells (MARC-145 cell line) were seeded in 96 well plate. Different concentration (10 ug-200 μg) of GRFT were added to the wells and incubated overnight. MTT solution was added and incubated for 3 hours at 370 C. 100 ul of MTT assay solvent was added and incubated in the dark for 15-20 mins. OD was measured at 590 nm. Cells without GRFT were used as controls (Li et al., Arch Virol. 2018; 163(12):3317-3325).

PRRSV virus was incubated at 37° C. with different concentration of GRFT and then added on to the target cells and incubated for 1 hr. After 1 hr. unbound virus was washed off and fresh medium was added and further incubated for 24 hrs. or 48 hrs. Infected cells was stained with 0.1% methylene blue solution and plaques were manually counted to establish PFU/ml (Plaque forming unit/ml) Or TCID 50 was calculated by Reed and Muench method (Lei et al., On the Calculation of TCID₅₀ for Quantitation of Virus Infectivity. Virol. Sin. (2020)).

To estimate the virus replication, infected cells were lysed for RNA isolation and QPCR was performed to quantify RNA of virus (Li et al., Arch Virol. 2018; 163(12):3317-3325).

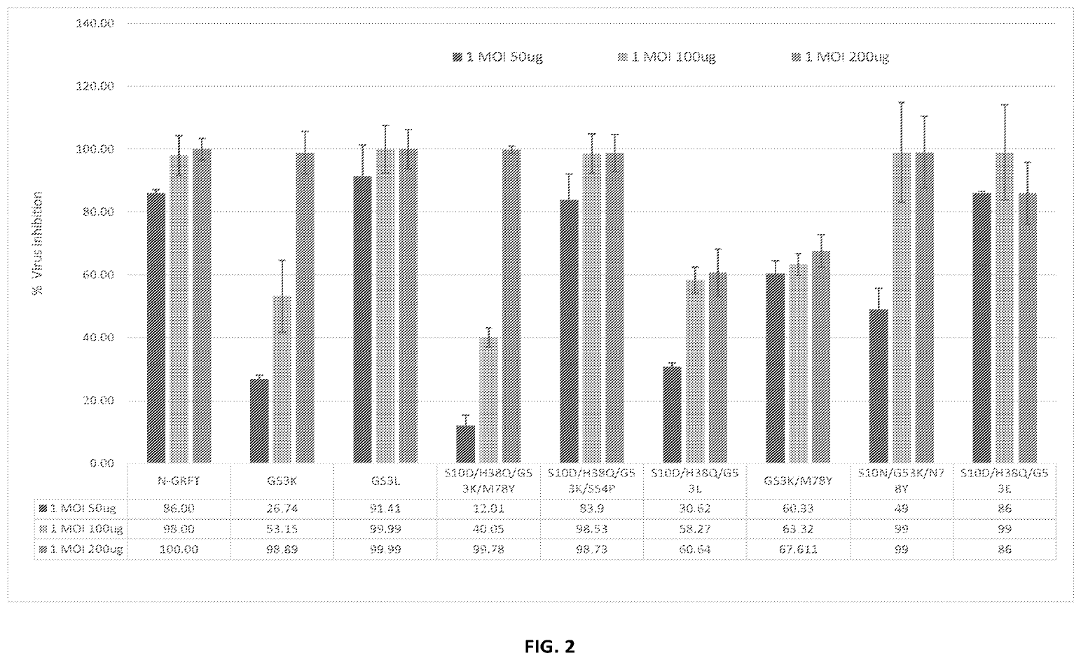

As shown in FIG. 2, nine Griffithsin variants were tested for their antiviral efficacy on MARC-145 cell line invested with low viral load of PRRSV. The single variants (M78N GRFT, G53K GRFT, G53L GRFT) and the combinatorial variants (GRFT S10D, H38Q, G53K, M78Y; GRFT S10D, H38Q, G53K, S54P; GRFT S10N, G35K, N78Y; GRFT S10D, H38Q, G53E) display the higher percentage of virus inhibition.

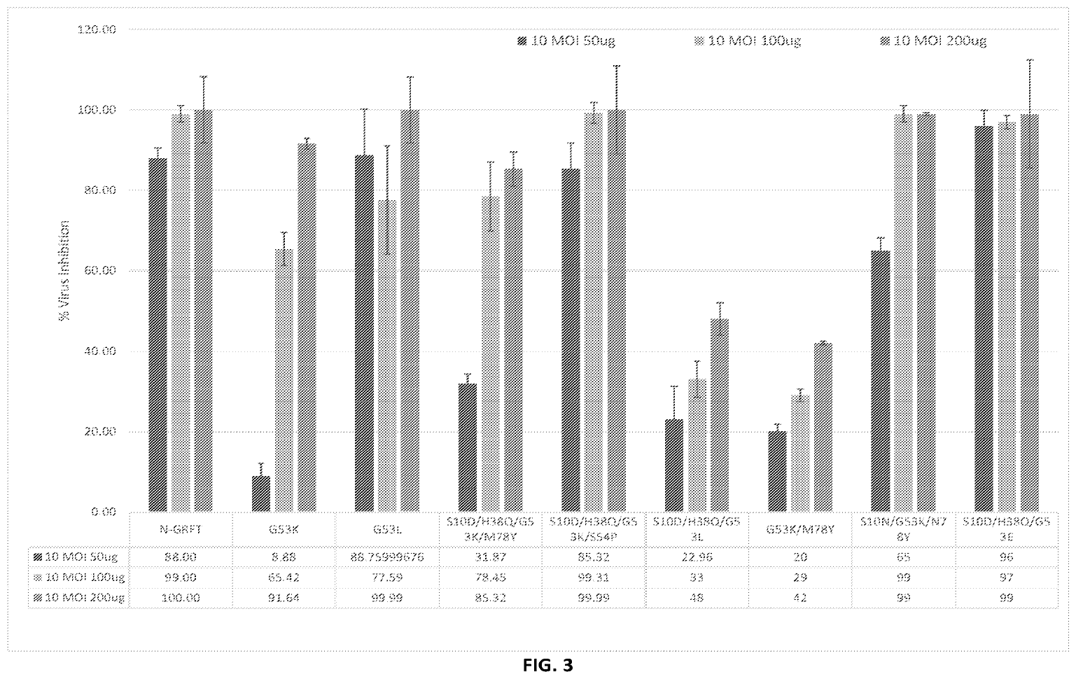

As shown in FIG. 3, nine Griffithsin variants were tested for their antiviral efficacy on MARC-145 cell line invested with high viral load of PRRSV. The single variants (M78N GRFT, G53K GRFT, G53L GRFT) and the combinatorial variants (GRFT S10D, H38Q, G53K, M78Y; GRFT S10D, H38Q, G53K, S54P; GRFT S10N, G35K, N78Y; GRFT S10D, H38Q, G53E) display the higher percentage of virus inhibition.

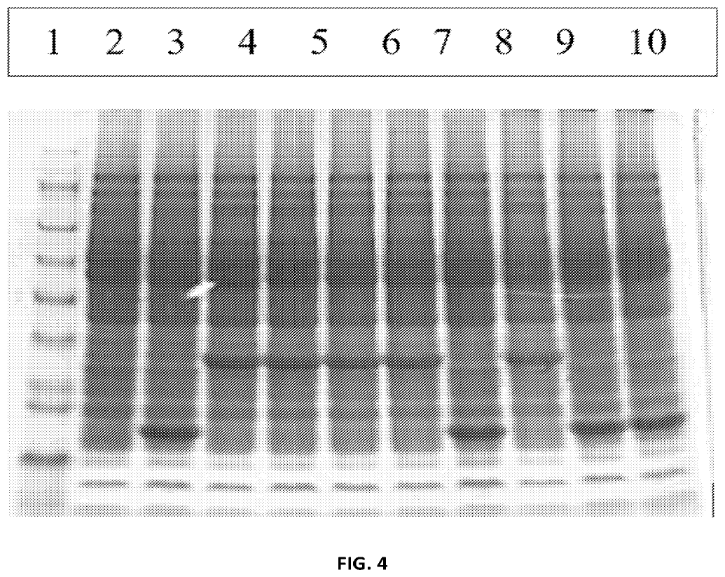

FIG. 4 depicts an SDS-PAGE gel of intracellular samples of Bacillus subtilis nine proteases deleted strains producing Griffithsin M78 monomer and dimers with GGG, GPG, GTG linkers. Lane 1 is See Blue Plus 2 molecular weight ladder; lane 2 is the negative control Bacillus subtilis nine proteases deleted strains; lanes 3, 8, 10 and 12 show the Griffithsin M78 monomer; lanes 4-5 are the Griffithsin M78 GGG dimers; lanes 6-7 are the Griffithsin M78 GPG dimers and lane 9 is the Griffithsin M78 GTG dimer.

### Example 5: Efficacy of the Griffithsin Nasal Spray Against PRRSV-2 (PRRSV 1-7-4 L1A Isolate) Virus

Fifty-six pigs (21 day old; mixed-sex, 1:1) were purchased and delivered to the animal facility at −7-day post challenge (−7 DPC). These pigs were screened to verify that they were virologically (tested by PCR) negative for PRRSV, swine influenza A virus, porcine circovirus 2 and 3 and serologically (tested by ELISA) negative for PRRSV.

Pigs were blocked by weight and then randomly divided into 4 groups: (1) NT/NC—Non treated non challenged pigs; (2) NT/C—Non treated challenged pigs; (3) LDT/C—Challenged pigs receiving low dose of Griffithsin; and (4) HDT/C—Challenged pigs receiving high dose of Griffithsin with 17 pigs per group, one room per each group (Table 3). The Griffithsin molecule used in this experiment corresponds to the dimer of SEQ ID NO:26. Each pig was microchipped for monitoring body temperature.

TABLE 3
Experimental design to study the effect of Griffithsin
administration on the PRRSV-2 virus infection.

| | | | Challenge2 -- | |
|---|---|---|---|---|
| Group¹ | | | 0 day post | |
| (number | Treatment | Antiviral treatment | challenge | Necropsy -- |
| of pigs) | description | (−3 DPC to 10 DPC) | (DPC) | 42 DPC |
| G1 | Untreated and | Mock-treated; | n = 05; sham | n = 5 |
| (n = 05) | Unchallenged | intranasal spray of PBS | challenge | |
| NT/NC | control | with carrier; twice daily | | |
| | | from −3 to 10 DPC | | |
| G2 | Untreated and | Mock-treated; | n = 17; PRRSV | n = 17 |
| (n = 17) | Challenged control | intranasal spray of PBS | challenge | |
| NT/C | | with carrier; twice daily | | |
| | | from −3 to 10 DPC | | |
| G3 | Antiviral treatment | Low dose antiviral | n = 17; PRRSV | n = 17 |
| (n = 17) | low dose + | treatment; intranasal | challenge | |
| LDT/C | Challenge | spray; twice daily | | |
| | 6 mg/pig/day | from −3 to 10 DPC | | |
| G4 | Antiviral treatment | High dose antiviral | n = 17; PRRSV | n = 17 |
| (n = 17) | high dose + | treatment; intranasal | challenge | |
| HDT/C | Challenge | spray; twice daily | | |
| | 12 mg/pig/day | from −3 to 10 DPC | | |

¹Treatment groups:
NT/NC—Non treated non challenged pigs;
NT/C—Non treated challenged pigs;
LDT/C—Challenged pigs receiving low dose of Griffithsin;
HDT/C—Challenged pigs receiving high dose of Griffithsin
2Challenge virus PRRSV 1-7-4 L1A isolate.~10⁵ TCID50/pig. 4 ml IN (2 ml per nostril)

From −3 through 10 DPC, Groups 1 and 2 were mock treated by receiving 1% Methocel E3 in phosphate buffered saline (PBS) via intranasal spray while Groups 3 and 4 received the antiviral compound at the corresponding dose via intranasal spray, twice daily. At 0 DPC, Group 1 received virus-negative medium as an unchallenged control whereas Groups 2 through 4 were challenged with a PRRSV 1-7-4 L1A isolate via intranasal (2 ml/nostril) inoculation at a dose of 10⁵ TCID50/pig. All pigs were euthanized and necropsied at 42 DPC.

Pigs were monitored daily for clinical signs including lethargy and anorexia. Microchip temperatures were recorded once daily in the morning. Pigs were weighed individually at −7, −3, 0, 10, 14, 21, 28, 35, and 42 DPC to calculate the average daily gain (ADG). Individual serum samples were collected at −4, 0, 1, 2, 4, 7, 10, 14, 21, 28, 35, and 42 DPC.

At the end of the study (42 DPC) or upon the death of the pigs during trial period, a necropsy was conducted for tissue sampling of all pigs. At necropsy, gross lung lesions were scored. A section of lung tissue was also placed in 10% formalin for histopathology evaluation.

Serum samples were tested by a quantitative PRRSV real-time RT-PCR. to determine the viremia level. Formalin-fixed lung tissues were examined for histopathological lesions and immunohistochemistry staining.

Clinical signs, body temperatures, average daily weight gain, virus levels in serum (viremia), gross lung lesion scores, microscopic lung lesions scores were compared between groups to assess the efficacy of the treatment.

For all outcome measures, data were analysed on a per-pig basis (except for oral fluid). Data were analysed by ANOVA, using the Fit Model platform of JMP 14.0. All viral load data were analysed on a log-transformed scale. Differences among treatment means were determined using Tukey's test. A probability of P<0.05 was considered significant and 0.05≤P<0.10 was considered as a tendency.

Preparation of Griffithsin nasal spray: 50 g of Methocel E3 Premium LV HP MC was dissolved in a total of 500 ml with PBS, this resulted 10% (w/v) Methocel E3 solution. Purified Griffithsin (KF474; corresponding to SEQ ID NO:26) was diluted to 16.7 g/L and 8.35 g/L for high dose and low dose respectively. One part of 10% Methocel E3 was added to nine parts of each diluted Griffithsin sample, which resulted in a final concentration of 1% Methocel E3 in PBS with 15 g/L Griffithsin or 7.5 g/L in the final formulation. They were mixed well before filtering through 0.22 um filter, and then were aliquoted and stored in −80° C.

### Results

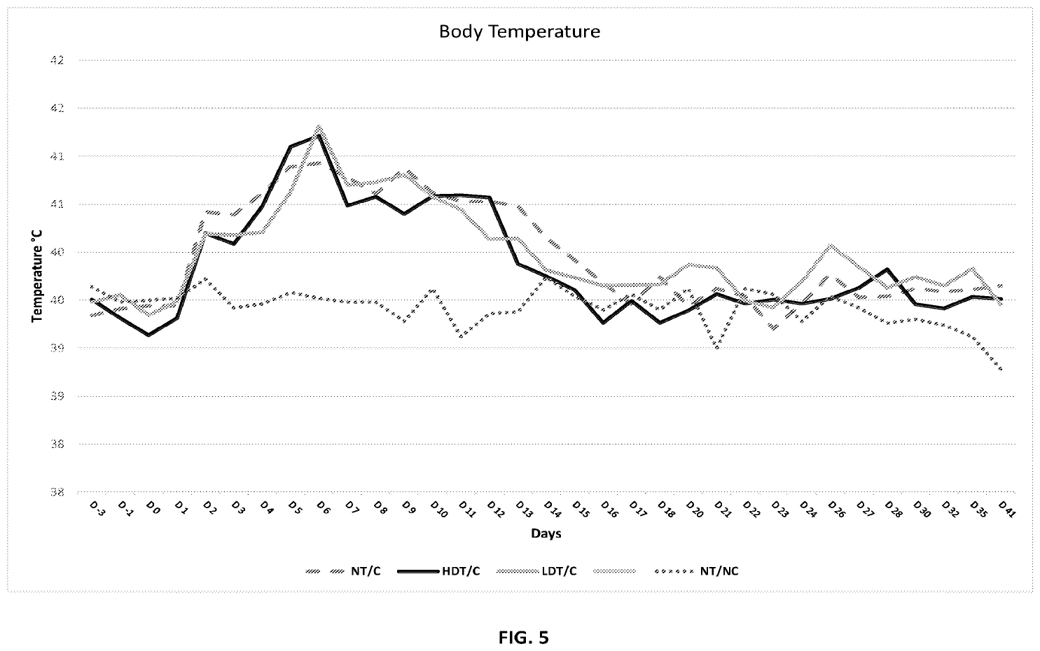

The PRRSV challenge model was successful in establishing viremia in pigs which was evident from the rise in body temperature in the challenged groups (FIG. 5). Moreover, pigs in HDT/C group returned to normal body temperature as early as d14 (DPI) as compared to day 23 for NT/C group.

Mortality was reduced in the Griffithsin-treated groups in comparison to the non-treated challenged pigs (12- and 6%-points in the LDT/C and HDT/C groups respectively when compared to NT/C) Table 4. No mortality was observed in the Non-challenged non-treated pigs (NT/NC).

TABLE 4
Mortality in pigs during 42-day post-challenge

| | Mortality (Number |
|---|---|
| Group¹ | of dead per group) |
| NT/NC | 0/5 |
| NT/C | 4/17 (23.6%) |
| LDT/C | 2/17 (11.8%) |
| HDT/C | 3/17 (17.6%) |

¹Treatment groups:
NT/NC—Non treated non challenged pigs;
NT/C—Non treated challenged pigs;
LDT/C—Challenged pigs receiving low dose of Griffithsin;
HDT/C—Challenged pigs receiving high dose of Griffithsin

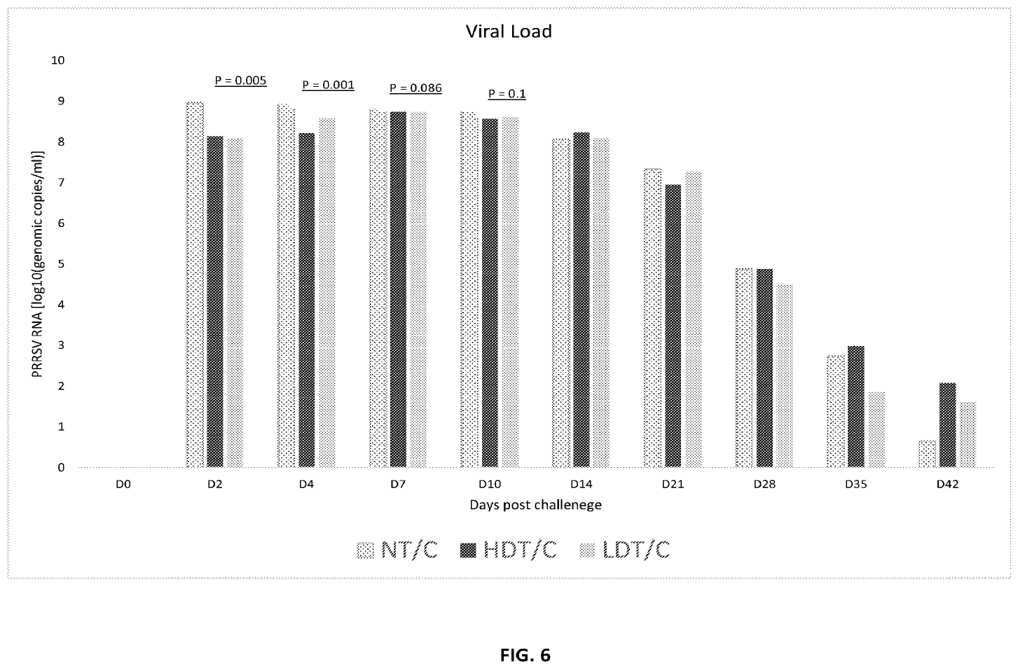

There was a significant (p=0.005, p=0.001) lower serum viral load on d 2 and 4 post-challenge and a tendency (p=0.1) for a reduction on 7 and 10 post-challenge in the HDT/C and for LDT/C group in comparison with NT/C group (FIG. 6).

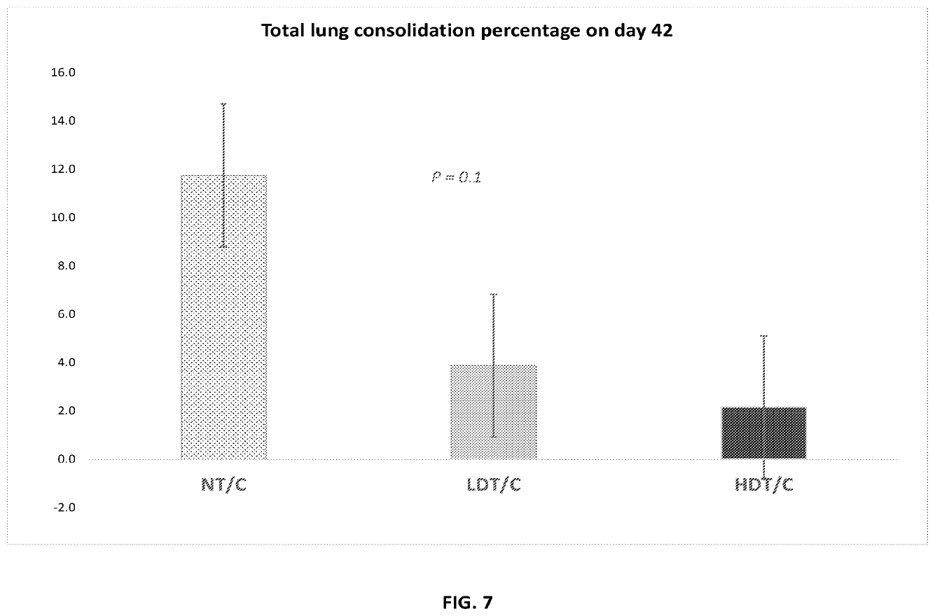

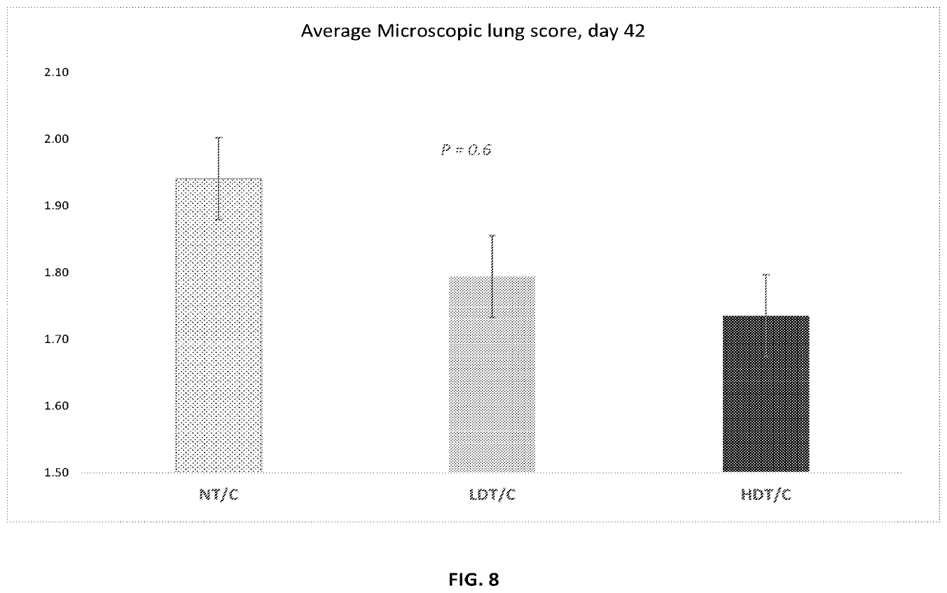

Both Griffithsin treated groups had milder gross lung lesions (tissue damage) compared to non-treated challenged pigs at the end of the study (FIG. 7) and relatively mild/moderate interstitial pneumonia (microscopic lesion) was observed in the lungs of Griffithsin treated pigs in comparison to the non-treated challenged pigs (FIG. 8).

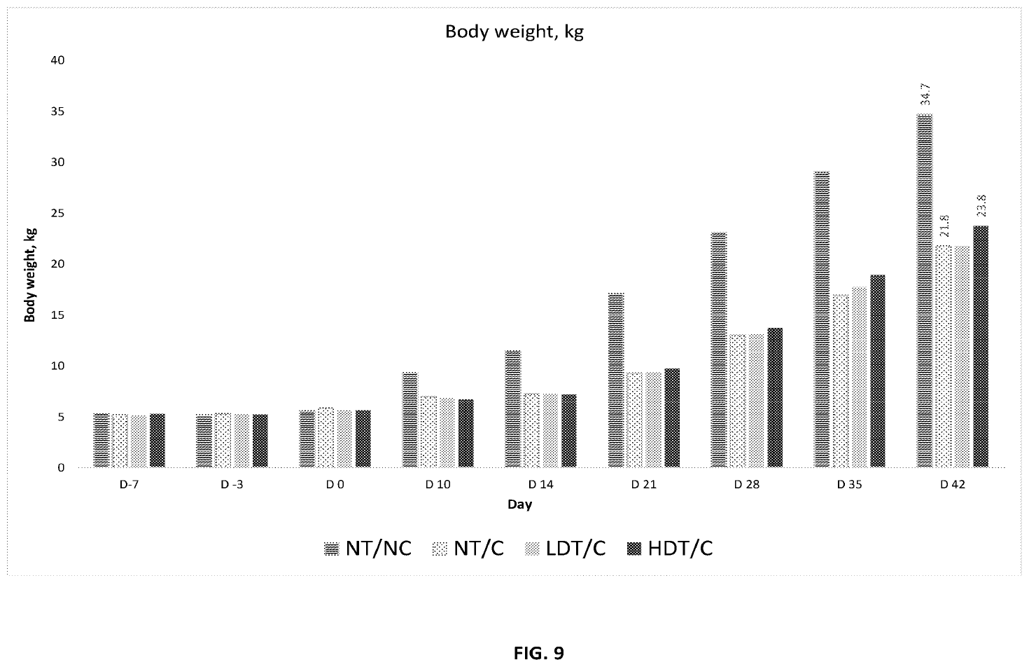

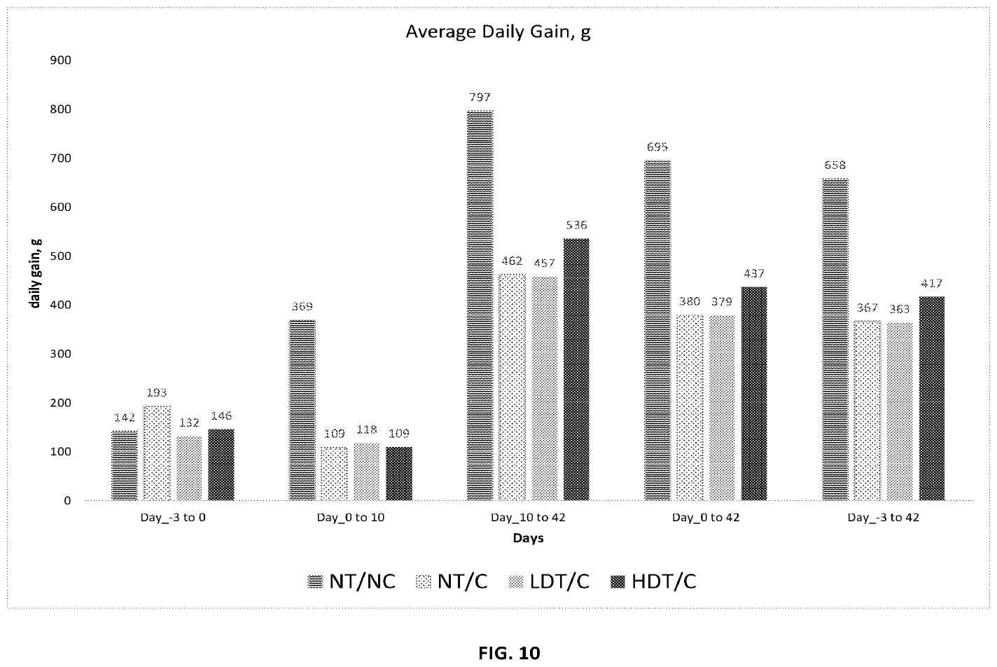

Regarding the growth performance, high dose of Griffithsin treatment resulted in a numerically higher final body weight and a tendency for a higher average daily gain compared to the non-treated challenged group (FIG. 9 & FIG. 10).

In conclusion, this example demonstrates that Griffithsin is an effective antiviral against PRRSV virus by the virtue of reduction in plasma viral load, reduction of the lesions in the lungs and better recovery form the infection by higher weight gain.

### REFERENCES

Albina et al., Veterinary Record 134(22):567-573, 1994.
Allende et al., Journal of Virology 74(22):10834-10837, 2000.
Ausubel et al., “Current Protocols in Molecular Biology,” published by Greene Publishing Assoc. and Wiley-Interscience (1987).
Ayouba et al., FEBS J, 289(1: 1-104, 1991
Benfield et al., Journal of Veterinary Diagnostic Investigation 4:127-133, 1992.
Breitenbach Barroso Coelho et al., J. Appl. Microbiol., 125(5): 1238-52, 2018,
Cole et al. J. Clinical Microbiol., 19(1): 48-54, 1984.
El-Araby et al., AMB Express, 10:90 (pages 1-14), 2020.
Gengenbach et al., Biotechnology and Bioengineering, 116, pages 2236-2249, 2019.
Hirayama et al., Mar. Biotechnol., Vol 18, pages 144-160, 2016.
Keffaber, Reproductive failure of unknown etiology. American Association of Swine Practitioners Newsletter 1:1-9, 1989.
Lagarda-Diaz et al., Int. J. Mol. Sci., 18, 1242, 2017.
Murtaugh et al., Archives of Virology 140(8):1451-1460, 1995.
Nelsen et al., Journal of Virology 73(1):270-280, 1999.
O'Keefe et al., PNAS, Vol 106, No. 15, pages 6099-6104, 2009.
Petrova et al., International Journal of Antimicrobial Agents, volume 52, issue 5, pages 599-607, 2018.
Petrova et al., Scientific Reports 6, Article No. 37437, 2016.
Peumans & Van Damme, Biotechnology and Genetic Engineering Reviews, 15:1, 199-228, 1998.
Pevzner et al., Biochimica et Biophysica Acta 1675: 155-164, 2004.
Ropp et al., Journal of Virology 78(7):3684-3703, 2001.
Sambrook et al., “Molecular Cloning: A Laboratory Manual” Cold Spring Harbor Laboratory: Cold Spring Harbor, N.Y. (1989), (2001) and (2012).
Singh & Sarathi, International J. Scientific & Engineering Res., Volume 3, Issue 4, 2012.
Wensvoort, et. al., Veterinary Quarterly 13:121-130, 1991.
Whitley et al., FEBS J., 280(9): 2056-206, 2013.
Wills et al., Veterinary Microbiology 55(1-4): 231-240, 1997.

We claim:

1. A non-naturally occurring variant lectin polypeptide or functional fragment thereof comprising an amino acid sequence at least about 60% identical to SEQ ID NO:8 or SEQ ID NO:18 comprising one or more substitutions at positions 10, 38, 53, 54, and 78.

2. The polypeptide of claim 1, comprising at least one substitution at position 10.

3. The polypeptide of claim 2, wherein the substitution at position 10 is selected from the group consisting of X10D, X10E, X10G, X10H, X10N, X10P, X10Q, and X10T.

4. The polypeptide of claim 3, wherein the substitution at position 10 is selected from the group consisting of S10D, S10E, S10G, S10H, S10N, S10P, S10Q, and S10T.

5. The polypeptide of any one of claims 1-4, comprising at least one substitution at position 38.

6. The polypeptide of claim 5, wherein the substitution at position 38 is selected from the group consisting of X38P and X38Q.

7. The polypeptide of claim 6, wherein the substitution at position A31 is selected from the group consisting of H38P and H38Q.

8. The polypeptide of any one of claims 1-7, comprising at least one substitution at position 53.

9. The polypeptide of claim 8, wherein the substitution at position 53 is selected from the group consisting of X53A, X53C, X53D, X53E, X53H, X53K, X53L, X53M, X53Q, X53R, X53T, and X53V.

10. The polypeptide of claim 9, wherein the substitution at position 53 is selected from the group consisting of G53A, G53C, G53D, G53E, G53H, G53K, G53L, G53M, G53Q, G53R, G53T, and G53V.

11. The polypeptide of any one of claims 1-10, comprising at least one substitution at position 54.

12. The polypeptide of claim 11, wherein the substitution at position 54 is selected from the group consisting of X54P and X54T.

13. The polypeptide of claim 12, wherein the substitution at position 54 is selected from the group consisting of S54P and S54T.

14. The polypeptide of any one of claims 1-13, comprising at least one substitution at position 78.

15. The polypeptide of claim 14, wherein the substitution at position 78 is selected from the group consisting of X78W, X78H, X78Q, and X78Y.

16. The polypeptide of claim 15, wherein the substitution at position 78 is selected from the group consisting of M/N78W, M/N78H, M/N78Q, and M/N78Y.

17. The polypeptide of any one of claims 1-16, wherein the polypeptide comprises at least two substitutions at positions 53 and 78.

18. The polypeptide of claim 17, wherein the substitution at position 53 is X53E, K, or L; and the substitution at position 78 is X78N or Y.

19. The polypeptide of claim 18, wherein the substitution at position 53 is G53E or K; and the substitution at position 78 is M/N78Y.

20. The polypeptide of any one of claims 1-19, wherein the polypeptide comprises at least three substitutions at positions 10, 53, and 78.

21. The polypeptide of claim 20, wherein the substitution at position 10 is X10D or N; the substitution at position 53 is X53K, E, or L; and the substitution at position 78 is X78Q or Y.

22. The polypeptide of claim 21, wherein the substitution at position 10 is S10D or N; the substitution at position 53 is G53K, E, or L; and the substitution at position 78 is M/N78Q or Y.

23. The polypeptide of any one of claims 1-19, wherein the polypeptide comprises at least three substitutions at positions 38, 54, and 78.

24. The polypeptide of claim 23, wherein the substitution at position 38 is X38Q; the substitution at position 54 is X54P; and the substitution at position 78 is X78Q.

25. The polypeptide of claim 24, wherein the substitution at position 38 is H38Q; the substitution at position 54 is S54P; and the substitution at position 78 is M/N78Q.

26. The polypeptide of any one of claims 1-19, wherein the polypeptide comprises at least three substitutions at positions 10, 38, and 53.

27. The polypeptide of claim 27, wherein the substitution at position 10 is X10N or D; the substitution at position 38 is X38Q; and the substitution at position 53 is X53E, L, or K.

28. The polypeptide of claim 28, wherein the substitution at position 10 is SION or D; the substitution at position 38 is H38Q; and the substitution at position 53 is G53E, L, or K.

29. The polypeptide of any one of claims 1-19, wherein the polypeptide comprises at least three substitutions at positions 10, 53, and 54.

30. The polypeptide of claim 27, wherein the substitution at position 10 is X10N or D; the substitution at position 53 is X53L or K; and the substitution at position 54 is X54P.

31. The polypeptide of claim 27, wherein the substitution at position 10 is SION; the substitution at position 53 is G53L; and the substitution at position 54 is S54P.

32. The polypeptide of any one of claims 1-31, wherein the polypeptide comprises at least four substitutions at positions 10, 38, 53, and 54.

33. The polypeptide of claim 32, wherein the substitution at position 10 is X10N or D; the substitution at position 38 is X38Q; the substitution at position 53 is X53E or K; and the substitution at position 54 is X54P.

34. The polypeptide of claim 33, wherein the substitution at position 10 is S10N or D; the substitution at position 38 is H38Q; the substitution at position 53 is G53E or K; and the substitution at position 54 is S54P.

35. The polypeptide of any one of claims 1-34, wherein the polypeptide comprises at least four substitutions at positions 10, 53, 54, and 78.

36. The polypeptide of claim 35, wherein the substitution at position 10 is X10N or D; the substitution at position 53 is X53L or K; the substitution at position 54 is X54P; and the substitution at position 78 is X78Y or Q.

37. The polypeptide of claim 36, wherein the substitution at position 10 is SION or D; the substitution at position 53 is G53L or K; the substitution at position 54 is S54P; and the substitution at position 78 is M/N78Y or Q.

38. The polypeptide of any one of claims 1-37, wherein the polypeptide comprises at least four substitutions at positions 10, 38, 53, and 78.

39. The polypeptide of claim 38, wherein the substitution at position 10 is X10D or N; the substitution at position 38 is X38Q; the substitution at position 53 is X53L, E, or K; and the substitution at position 78 is X78Y or Q.

40. The polypeptide of claim 39, wherein the substitution at position 10 is S10D or N; the substitution at position 38 is H38Q; the substitution at position 53 is G53L, E, or K; and the substitution at position 78 is M/N78Y or Q.

41. The polypeptide of any one of claims 1-40, wherein the polypeptide comprises at least five substitutions at positions 10, 38, 53, 54, and 78.

42. The polypeptide of claim 41, wherein the substitution at position 10 is X10N or D; the substitution at position 38 is X38Q; the substitution at position 53 is X53K or L; the substitution at position 54 is X54P; and the substitution at position 78 is X78Y or Q.

43. The polypeptide of claim 41, wherein the substitution at position 10 is S10N or D; the substitution at position 38 is H38Q; the substitution at position 53 is G53K or L; the substitution at position 54 is S54P; and the substitution at position 78 is M/N78Y or Q.

44. The polypeptide of any one of claims 1-43, wherein the variant lectin polypeptide or functional fragment thereof is capable of naturally dimerizing with other lectin polypeptides.

45. A polypeptide dimer comprising two polypeptides or functional fragments thereof comprising an amino acid sequence at least about 60% identical to SEQ ID NO:8 or SEQ ID NO:18.

46. A variant lectin polypeptide dimer comprising two polypeptides or functional fragments thereof comprising any of the polypeptides of claims 1-44.

47. The polypeptide dimer of claim 45 or claim 46, wherein the two polypeptides in the dimer are identical.

48. The polypeptide dimer of any one of claims 45-47, wherein the two polypeptides in the dimer are separated by a linker amino acid sequence.

49. The polypeptide dimer of claim 48, wherein the linker is between 2 and 50 amino acids in length.

50. The polypeptide dimer of claim 48 or claim 49, wherein the linker comprises the amino acid sequence (GTG)ₙ, where n=1-7.

51. The polypeptide dimer of claim 48 or claim 49, wherein the linker comprises (i) any one of SEQ ID NOs:32-57; or (ii) (GGG)ₙ or (GPG)ₙ, where n=1-7.

52. The polypeptide dimer of any one of claims 45-51, wherein each polypeptide of the polypeptide dimer is capable of dimerization with a Griffithsin protein.

53. The polypeptide dimer of any one of claims 45-52, wherein the dimer comprises an amino acid sequence selected from the group consisting of SEQ ID NO:20, SEQ ID NO:22, SEQ ID NO:24, SEQ ID NO:26, SEQ ID NO:29, and SEQ ID NO:31.

54. The polypeptide of any one of claims 1-53, wherein the polypeptide has antiviral activity.

55. A nucleic acid encoding the polypeptide of any one of claims 1-54.

56. A vector comprising the nucleic acid of claim 55.

57. A recombinant host cell comprising the nucleic acid of claim 55 or the vector of claim 56.

58. The host cell of claim 56, wherein the host cell is a fungal cell, an algal cell, a plant cell, a bacterial cell, or a yeast cell.

59. The host cell of claim 58, wherein the host cell is a Bacillus subtilis cell.

60. A pharmaceutical composition comprising a therapeutically effective amount of the polypeptide of any one of claims 1-53 and a pharmaceutically acceptable excipient.

61. The pharmaceutical composition of claim 60, wherein the composition is formulated for oral, nasal, and/or topical administration to an animal.

62. A method for treating or preventing a viral infection in an animal, the method comprising administering the polypeptide of any one of claims 1-54 or the pharmaceutical composition of any one of claims 59-61 to the animal.

63. The method of claim 62, wherein the polypeptide or pharmaceutical composition is administered to the animal via oral, nasal, and/or topical administration.

64. The method of claim 62 or claim 63, wherein the animal is swine or a horse.

65. The method of claim 64, wherein the swine is a sow, gilt, boar, lactation-phase piglet, weaned piglet, and/or finishing pig.

66. The method of any one of claims 62-65, wherein the viral infection comprises the virus that causes porcine reproductive and respiratory syndrome (PRRSV), porcine epidemic diarrhea virus (PEDV), porcine rotavirus, or equine viral arteritis (EVA).

67. A method for producing a polypeptide comprising culturing the host cell of any one of claims 57-59 in a suitable media under conditions suitable for polypeptide expression.

68. The method of claim 67, further comprising purifying the polypeptide.
